(19) United States
(12) Patent Application Publication
(10) Pub. No.: US 2017/0348398 A1
(43) Pub. Date: Dec. 7, 2017

(54) COMPOSITIONS AND METHODS FOR DECREASING BLOOD GLUCAGON LEVELS

(71) Applicant: Vanderbilt University, Nashville, TN (US)

(72) Inventors: Alvin C. Powers, Brentwood, TN (US); Erika Danielle Dean, Nashville, TN (US)

(21) Appl. No.: 15/612,799

(22) Filed: Jun. 2, 2017

Related U.S. Application Data
(60) Provisional application No. 62/345,690, filed on Jun. 3, 2016.

(51) Int. Cl. A61K 38/50; A61K 45/06; C12N 5/071; A61K 31/196; A61K 31/655; A61K 31/433; A61K 31/198; G01N 33/50; A61K 31/192

(52) U.S. Cl. CPC A61K 38/50; G01N 33/507; C12Y 305/01001; A61K 45/06; C12N 5/0676; A61K 31/192; A61K 31/655; A61K 31/433; A61K 31/198; A61K 31/196; C12N 2500/32

(57) ABSTRACT

Disclosed are improved compositions and methods for decreasing blood glucagon levels. As disclosed herein, L-glutamine is a selective stimulator of α-cell proliferation generated when glucagon signaling is interrupted. Therefore, disclosed is a method for treating a subject with hyperglucagonemia, e.g., a subject with diabetes, that involves administering to the subject a composition comprising an L-glutamine inhibitor in an amount effective to decrease blood glucagon levels.

This application claims the benefit of U.S. Provisional Application No. 62/345,690, filed Jun. 3, 2016, which is incorporated herein by reference in its entirety.

### STATEMENT REGARDING FEDERALLY SPONSORED RESEARCH OR DEVELOPMENT

This invention was made with Government Support Grant Nos. DK07563, DK020593, and DK66636 awarded by the National Institutes of Health. The Government has certain rights in the invention.

### BACKGROUND

Blood glucose homeostasis is regulated primarily as a result of coordinated secretion of two pancreatic islet-derived hormones, insulin and glucagon. Hypoglycemia and amino acids stimulate glucagon secretion from α-cells in the pancreatic islet. Glucagon in turn stimulates glucose production via gluconeogenesis and glycogenolysis in the liver. Diabetes has been traditionally thought to result from impaired insulin action leading to reduced uptake of glucose by insulin-sensitive tissues and hyperglycemia. More recently the contribution of absolute or relative hyperglucagonemia relative to the hyperglycemia of Type 1 and Type 2 diabetes has been recognized. Consequently, efforts to reduce glucagon action using small molecule antagonists, siRNA, aptamers, or antibodies that target the glucagon receptor (Gcgr) have successfully improved glycemic control, especially in type 2 diabetes. However, interruption of glucagon signaling by multiple approaches (proglucagon gene knockout, interruption of Gcgr or its signaling, Gcgr small molecule inhibitors, Gcgr antibodies, or Gcgr antisense oligos nucleotides) results in hyperglucagonemia and α-cell hyperplasia.

### SUMMARY

In one aspect, disclosed herein are methods for treating a subject with diabetes. In one aspect, said treatment methods can comprise administering to the subject a composition comprising an L-glutamine inhibitor in an amount effective to decrease blood glucagon levels.

Also disclosed are methods of any preceding aspect, wherein the L-glutamine inhibitor is a glutaminase (GLS) inhibitor (such as, for example, Bis-2-(5-phenylacetamido-1,3,4-thiadiazol-2-yl)ethyl sulfide (BPTES); azaserine; 6-diazo-5-oxo-L-norleucine (L-DON); an inhibitor of a SLC7A5/SLC3A2 transporter (such as, for example, 2-aminobicyclo-(2,2,1) heptanecarboxylic acid (BCH)); an inhibitor of SLC1A5 transporter (such as, for example, L-γ-glutamyl-p-nitroanilide (GPNA)); 4-Phenylbutyrate (4-PBA) and/or an Asparaginase (such as, for example, Asparaginase Medac, Ciderolase, ONCASPAR®, ERWINASE®, ELSPAR®).

In one aspect, disclosed herein are methods of any preceding aspect, wherein the subject has pre-diabetes, type 1 diabetes, type 2 diabetes, or gestational diabetes.

Also disclosed are methods of any preceding aspect, wherein the L-glutamine inhibitor is administered in combination with one or more of an arginine inhibitor, metaformin, or insulin, a GLP-1 agonist, and/or DDP-4 inhibitor.

Also disclosed herein are methods for the screening of a pancreatic-alpha-cell proliferation inducing compound, comprising the steps of a) contacting at least one pancreatic alpha-cell with a given compound, and b) testing whether said compound is capable of ki67 or pHH3 gene or protein expression.

In one aspect, disclosed herein are methods for the screening of a pancreatic-alpha-cell proliferation inducing compound, comprising the steps of a) contacting at least one pancreatic alpha-cell with a given compound, and b) testing whether said compound is capable Edu or BrdU incorporation.

Also disclosed herein are methods for expanding alpha cells in culture, comprising contacting the alpha cells with an effective amount of a composition comprising L-glutamine.

In one aspect, disclosed herein are methods of expanding alpha cells of any preceding aspect, further comprising transdifferentiating the expanded alpha cells into beta cells.

Also disclosed are alpha cell expansion methods of any preceding aspect, further comprising transplanting the beta cells into a subject with diabetes. In one aspect, the alpha cells and/or beta cells can be autologous to the subject.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A, 1B, 1C, 1D, 1E, 1F, and 1G show that α-cells proliferate in vitro in response to Glucagon receptor (Gcgr)−/− mouse serum. FIG. 1A shows a schematic for in vitro α-cell proliferation assay to quantify proliferation rates. Representative images of dispersed islet cells cultured in (B) control media, (C) Gcgr+/+ mouse serum-supplemented media or (D) Gcgr−/− mouse serum-supplemented media. Glucagon staining (green), Ki67 staining (red), and DAPI staining (blue) are shown. Proliferating α-cells (Ki67⁺ glucagon⁺) are indicated by white arrows. Proliferating non α-cells (Ki67⁺ glucagon⁻) are indicated by yellow arrows. Inset provides higher magnification of both proliferating α- and non-α-cells. White bar indicates 75 μm. (E) Quantification of α-cell proliferation following culture in mouse serum-supplemented media. Control media with no mouse serum added (grey bars, n=12 experiments), 10% Gcgr+/+ mouse serum treated (white bar, n=3 experiments), and 10% Gcgr−/− mouse serum treated (red bar, n=12 experiments experiments) are shown. α-cell proliferation (Ki67⁺ α cells %) is defined by the percentage of total number of glucagon⁺Ki67⁺ double-positive cells per total number glucagon⁺ cells. ***p<0.001 vs control media, ^{#}p<0.05 vs. Gcgr+/+ mouse serum treated-islets. (F) Quantification of Gcgr−/− size fractionated mouse serum induced α-cell proliferation in cultured mouse islets. Control media with no mouse serum added (grey bars, n=13 experiments), 10% Gcgr−/− whole mouse serum-supplemented media (red bar, n=12 experiments), 10% Gcgr−/−<10 kDa mouse serum-supplemented media (red left hashed bar, n=7 experiments), and 10% Gcgr−/−<10 kDa mouse serum-supplemented media (red right hashed bar, n=6 experiments) islets are shown. ***p<0.001 vs control media, ^{##}p<0.01 vs. Gcgr−/− mouse serum treated-islets, and ^{$$}p<0.01 vs. <10 kDa Gcgr−/− mouse serum-supplemented media. (G) Quantification of percentage of cells proliferating in each fractionated serum-supplemented media condition that are α-cells (the total number of glucagon⁺ Ki67⁺ double positive cells per the total number of Ki67⁺ cells). Control media with no mouse serum added (grey bars, n=7), Gcgr−/− whole mouse serum-supplemented media (red bar, n=6), Gcgr−/−<10 kDa mouse serum-supplemented media (red left hashed bar, n=3), and Gcgr−/−<10 kDa mouse serum-supplemented media (red right hashed bar, n=3) islets are shown. ***p<0.001 vs control media, ^{##}p<0.01 vs. Gcgr−/− mouse serum-supplemented media, and ^{$$}p<0.01 vs. <10 kDa Gcgr−/− mouse serum-supplemented media.

FIGS. 2A, 2B, 2C, 2D, 2D′, 2E, 2E′, 2F, 2F′, 2G, 2G′, 2H, 2I, and 2J show that the molecular target of rapamycin (mTOR) signaling and FoxP transcription factor are essential for α-cell proliferation in response to interrupted glucagon signaling. (A) Fasting blood glucose (mg/dl) n=5, (B) fasting serum glucagon (μg/ml) n=5, and (C) α-cell proliferation (n=3) in mice after cotreatment with Gcgr mAb and rapamycin. Saline/phosphate buffered saline (PBS) treated (white bars), Saline/Gcgr mAb treated (blue bars), Rapamycin/PBS treated (white left hashed bars) and rapamycin/Gcgr mAb treated (blue left hashed bars) are shown. *p<0.05, **p<0.01, and ***p<0.001 vs PBS treated and ^{#}p<0.05, ^{##}p<0.01, and ^{###}p<0.001 vs. Saline treated. (D-E) Representative images of pancreatic islet α-cell proliferation in Saline/Gcgr mAb- and Rapamycin/Gcgr mAb-treated mice. Glucagon staining (green), Ki67 staining (red), and DAPI staining (blue) are shown. White scale bars indicate 100 μm. White dashed boxes indicate region selected for insets (D′-E′). (F-G) Representative images of pancreatic islet α-cell expression of pS6 protein in Saline/Gcgr mAb- and Rapamycin/Gcgr mAb-treated mice. Glucagon staining (green), pS6(pS235/S236) staining (red), and DAPI staining (blue) are shown. White scale bars indicate 100 μm. White dashed boxes indicate region selected for insets (F′-G′). (H) α-cell number in 7 days post-fertilization (dpf) wildtype and GcgR1^{-/-} GcgR2^{-/-} zebrafish larvae primary islet after treatment with rapamycin for 3 days. Wildtype/vehicle treated (white bars, n=8), GcgR1^{-/-}GcgR2^{-/-}/vehicle treated (green bars, n=8), Wildtype/Rapamycin treated (white left hashed bars, n=9) and GcgR1^{-/-}GcgR2^{-/-}/Rapamycin treated (green left hashed bars, n=11) are shown. *p<0.05 and ***p<0.001 vs Wildtype and ^{#}p<0.05 vs. vehicle treated. (I) α-cell proliferation in rapamycin and 10% Gcgr−/− mouse serum co-supplemented media cultured mouse islets. Control media (plus vehicle) with no mouse serum added (grey bars, n=3), Gcgr−/− whole mouse serum (plus vehicle)-supplemented media (red bar, n=2) and Gcgr−/− mouse serum and rapamycin co-supplemented media cultured mouse islets (red left hashed bar, n=3) are shown. **p<0.01 vs control media and ^{#}p<0.05 and ^{##}p<0.01 vs. Gcgr−/− mouse serum (plus vehicle)-supplemented media. (J) α-cell proliferation in Gcgr mAb-treated FoxP1/2/4^{-/-} mice is shown. Wildtype/PBS-treated (white bars, n=3), Wildtype/Gcgr mAb-treated (blue bars, n=3), FoxP1/2/4^{-/-}/PBS-treated (white left hashed bars, n=3) and FoxP1/2/4^{-/-}/Gcgr mAb-treated (blue left hashed bars, n=3) are shown. ***p<0.001 vs PBS-treated and ^{##}p<0.01 vs. Saline-treated.

FIGS. 3A, 3B, 3C, and 3D show that acute and chronic models of interrupted glucagon receptor signaling have common alterations in liver gene expression. (A) Schematic of systems biology strategy to identify hepatic factor stimulating α-cell proliferation. (B) Venn diagram of gene changes in liver of mice. (C-D) Gene ontology analyses reveal enrichment of pathways related to lipid (blue bars) and amino acid metabolism (red bars) in (C) Gcgr mAb (gray bars) and (D) Gcgr−/− (black bars) versus Gcgr+/+ mice.

FIGS. 4A, 4B, 4C, 4D, 4E, and 4F show that alterations in lipid and bile acid metabolism and secreted factors in models with interrupted glucagon receptor signaling. (A) Log₂ fold changes in liver gene expression related to lipid and bile metabolism. Gcgr mAb-treated Gcgr+/+ versus PBS-treated Gcgr+/+ mice (blue bars, n=3) and PBS-treated Gcgr−/− versus PBS-treated Gcgr+/+ mice (red bars, n=3) are shown. (B) Serum bile acid levels in PBS-treated Gcgr+/+ (white bars), Gcgr mAb-treated Gcgr+/+ (blue bars), and PBS-treated Gcgr−/− (red bars) mice. One-way ANOVA with Tukey posthoc analyses; *p<0.05, ***p<0.001 vs Gcgr−/− mouse serum. (C) α-cell proliferation in response to delipidated Gcgr−/− mouse serum. Control media with no mouse serum added (grey bars, n=2), 10% Gcgr−/− whole mouse serum-supplemented media (red bar, n=2), 10% Gcgr−/− delipidated mouse serum-supplemented media (red left hashed bar, n=2) islet cultures are shown. One-way ANOVA with Tukey posthoc analyses; *p<0.05 vs control media. (D) Log₂ fold changes in liver gene expression of predicted secreted proteins. (E) Plot of top gene changes in each model. Log₂ fold gene changes observed in both Gcgr+/+ vs. Gcgr−/− and Gcgr+/+ vs. Gcgr mAb-treated mice are shown with green circles. Log₂ fold gene changes observed in both Gcgr+/+ vs. Gcgr−/− and WT vs. Gcg+ mice (see Song et al., 2014 for details on WT vs. Gcg+ mice) are shown with blue triangles. Black arrows indicate gene expression changes in three secreted factors. (F) Manhattan Plots of proteins significantly altered in serum of mice with “chronic” Gcgr−/− PBS-treated versus Gcgr+/+ PBS-treated mice.

FIGS. 5A, 5B, 5C, 5D, 5E, 5F, 5G, 5H, 5I, 5J, and 5K show that L-glutamine and other amino acids promote selective α-cell proliferation. (A) Log₂ fold changes in expression of liver amino acid catabolism genes in Gcgr−/− (red bars, n=3) and Gcgr mAb (blue bars, n=3)-treated mice relative to PBS-treated Gcgr+/+ mice. (B) Serum amino acid levels in Gcgr−/− (red bars, n=3), Gcgr mAb (blue bars, n=5)-treated, and PBS-treated (white bars, n=8) *p<0.05, **p<0.01, ***p<0.001 vs PBS-treated Gcgr+/+ mice. #p<0.05, ##p<0.01 vs. Gcgr mAb-treated mice. (C) Quantification of α-cell proliferation in response to media with increasing amino acid concentration containing media for 3 days. This is the total number of glucagon⁺ Ki67⁺ double positive cells per the total number of glucagon⁺ cells. White (low, n=3) to gray (intermediate, n=3-8) to black (high, n=7) color indicates total concentration of all amino acids in each media condition. The red bar indicates 10% Gcgr−/− serum-supplemented media (n=6) similar as in FIG. 1E. (D) Quantification of percentage of cells proliferating under each amino acid condition that are α-cells in cultured mouse islets treated for 3 days. This is the total number of glucagon⁺ Ki67⁺ double positive cells per the total number of Ki67⁺ cells. Control media with no mouse serum added (grey bars), Gcgr−/− whole mouse serum treated (red bar), White (low) to gray (intermediate) to black (high) color indicates concentration of collective amino acids in each media condition. The red bar contains 10% Gcgr−/− serum similar as in FIG. 1G. One-way ANOVA with Tukey posthoc analyses; ***p<0.001 vs Gcgr−/− mouse serum-supplemented media (red bar), ^{###}p<0.01 vs. highest amino acid containing media-treated islets (black bar), and ^{$}p<0.05 vs. highest amino acid containing media-treated islets (dark charcoal bar). (E) Linear regression analyses of amino acid concentration in each media condition versus the α-cell proliferation rate with each media. Significant correlation related to glutamic acid (blue triangles), glutamine (red squares) and leucine (green triangles) concentrations are noted. (F) Quantification of α-cell proliferation and (G) percentage of cells proliferating that are α-cells in response to altering individual amino acid levels in cultured mouse islets. One-way ANOVA with Tukey posthoc analyses; n=3, ***p<0.001 vs high L-glutamate, L-leucine, and L-glutamine media (High Glutamate Leucine Glutamine (ELQ))-treated islets, ^{###}p<0.001 vs. low L-glutamate (Low E)-treated islets, and ^{$$$}p<0.001 vs. low L-leucine (Low L)-treated islets. (H) Quantification of L-glutamine dose response stimulated α-cell proliferation and (I) percentage of cells proliferating that are α-cells in cultured mouse islets treated for 3 days. One-way ANOVA with Tukey posthoc analyses; n=3, ***p<0.001 vs 3250 μM L-glutamine media-treated islets, ^{##}p<0.01 vs 2055 μM L-glutamine media-treated islets. (J) Quantification of rapamycin effects on amino acid-stimulated α-cell proliferation and (K) percentage of cells proliferating that are α-cells in cultured mouse islets treated for 3 days. One-way ANOVA with Tukey posthoc analyses; highest amino acid media with DMSO added (black bars n=3), highest amino acid media with 30 nM rapamycin added for the last 24 hours of culture (black left hashed bar, n=2), highest amino acid media with 30 nM rapamycin added for the full 72 hours of culture (black right hashed bar, n=3).

FIGS. 6A, 6B, 6C, 6D, 6E, 6F, 6G, and 6H show that human pancreatic islet α-cells proliferate when glucagon signaling is interrupted. (A) Schematic of experimental design for human islet subcapsular renal transplantation and administration of Gcgr mAb. (B) Fasting blood glucose (n=49-50), (C) fasting serum glucagon levels (n=42-55), and (D) fasting serum GLP-1 levels on Day 10 in mice treated with either PBS (grey circles) or Gcgr mAb (grey triangles). (Student's t-test, ****p<0.0001; N.D. not detected). Note: Fasting serum GLP-1 levels of all PBS-treated mice (n=10) were below the detection limit of the GLP-1 assay (41 pg/ml). The GLP-1 level in one fasting Gcgr mAb mouse sample was below the assay's detection limit. Therefore, no statistical analyses were performed. Quantification of (E) mouse α-cell proliferation (n=3) and (F) human islet donor (Donor 4) graft α-cell proliferation (n=5-6) 2 weeks after treatment with Gcgr mAb. (G) Quantification of human islet α-cell proliferation in individual transplanted donor islets grafts from 7 different experiments of NOD-scid-gamma (NSG) mice treated with PBS or Gcgr mAb for 2 to 6 weeks. (H) Model for liver-pancreatic islet α-cell axis where L-glutamine and glucagon reciprocally regulate each other. Glucagon is released from the pancreatic islet α-cell where it acts on Gcgrs on hepatocytes to stimulate gluconeogenesis, and hepatic glucose output raising blood glucose. When glucagon signaling is interrupted in hepatocytes, this leads to impaired gluconeogenesis, decreased amino acid catabolism, and increased circulating amino acids. Of these amino acids, L-glutamine selectively activates α-cell proliferation through mTOR and FoxP-dependent mechanisms. Glutaminase 2-GLS2.

FIGS. 7A, 7B, 7C, 7D, and 7E show that Gcgr−/− mice have severe α-cell hyperplasia. (A-B) Representative images of pancreatic α-cell staining in Gcgr+/+ (left) and Gcgr−/− (right) 5 month old mice. Glucagon staining (green), amylase staining (red), and DAPI staining (blue); White boxes indicate regions for insets. White scale bars indicate 1 mm. (C) Schematic for in vitro α-cell proliferation assay with algorithm building to measure proliferation rates. (D) Dose responsiveness of α-cell proliferation in mouse to Gcgr−/− mouse serum-supplemented media. Control media with no mouse serum added (grey bars, n=3) and increasing doses of Gcgr−/− whole mouse serum-supplemented media (red bars, n=2-8) are shown. **p<0.01 vs control media, ^{##}p<0.01 vs. 0.1% Gcgr−/− mouse serum-supplemented media, and ^{$}p<0.05 vs. 1% Gcgr−/− mouse serum-supplemented media. (E) Quantification of α-cell proliferation in mouse Gcgr−/− hepatocyte conditioned media-treated mouse islets. Control media with no conditioned media added (grey bars, n=5), control hepatocyte with unconditioned media added (grey with black left hash bar, n=2) and Gcgr−/− hepatocyte conditioned media-treatment added (red with grey hash bar, n=4) are shown. One-way ANOVA with Tukey posthoc analyses; *p<0.05 vs control media.

FIGS. 8A, 8B, 8C, 8D, 8D′, 8E, 8E′, 8F, 8F′, 8G, 8G′, 8H, 8I, and J show that mTOR signaling and FoxP transcription factor are essential for α-cell proliferation in response to interrupted glucagon signaling. (A) Random blood glucose (mg/dl), (B) glucose/arginine-stimulated blood glucose (mg/dl), and (C) glucose/arginine-stimulated serum glucagon (pg/ml) in mice after cotreatment with Gcgr mAb and rapamycin. Saline/PBS treated (white bars), Saline/Gcgr mAb treated (blue bars), Rapamycin/PBS-treated (white left hashed bars) and rapamycin/Gcgr mAb treated (blue left hashed bars) are shown. Two-way ANOVA with Bonferroni posthoc analyses; *p<0.05, **p<0.01, and ***p<0.001 vs PBS treated and ^{#}p<0.05 and ^{###}p<0.001 vs. Saline treated. (D-E) Representative images of pancreatic islet α-cell proliferation in Saline/PBS- and Rapamycin/PBS-treated mice. Glucagon staining (green), Ki67 staining (red), and DAPI staining (blue) are shown. White scale bars indicate 100 μm. White dashed boxes indicate region selected for insets (D′-E′). (F-G) Representative images of pancreatic islet α-cell expression of pS6 protein in Saline/PBS- and Rapamycin/PBS-treated mice. Glucagon staining (green), pS6(pS235/S236) staining (red), and DAPI staining (blue) are shown. White scale bars indicate 100 μm. White dashed boxes indicate region selected for insets (F′-G′). (H) Body mass, (I) random blood glucose (mg/dl), and (J) pancreatic mass in Gcgr mAb-treated FoxP1/2/4^{-/-} mice. Wildtype/PBS-treated (white bars), Wildtype/Gcgr mAb-treated (blue bars), FoxP1/2/4^{-/-}/PBS-treated (white left hashed bars) and FoxP1/2/4^{-/-}/Gcgr mAb-treated (blue left hashed bars) are shown. Two-way ANOVA with Bonferroni posthoc analyses; *p<0.05 and ***p<0.001 vs PBS-treated and ^{##}p<0.01 vs. Saline-treated.

FIGS. 9A, 9B, 9C, 9D, 9E, 9F, 9G, 9H, 9I, 9J, and 9K show that acute and chronic models of interrupted glucagon receptor signaling have common alterations in liver gene expression. (A) Body mass (g), (B) random blood glucose (mg/dl), (C) pancreas mass, and (D) α-cell proliferation in mice treated with PBS or Gcgr mAb for 10 days. (E-F) Representative images of pancreatic islet α-cell proliferation in mice treated with PBS or Gcgr mAb for 10 days. Glucagon staining (green), Ki67 staining (red), and DAPI staining (blue) are shown. White arrows indicate proliferating α-cells. (G) Fasting blood glucose in PBS-treated Gcgr+/+ (brown), Gcgr mAb-treated (blue) or PBS-treated Gcgr−/− (red) mice. (H) Principle component analysis of liver RNA-Seq from mice with interrupted glucagon signaling. (I) Spearman correlation of each treatment group from RNA-Seq analyses. Volcano plots of genes altered in RNA-Seq analyses of livers from (J) “acute” Gcgr mAb treatment and (K) “chronic” Gcgr−/− mice versus Gcgr+/+ mice. Red dots are genes that are significantly downregulated in either Gcgr mAb or Gcgr−/−mice (p<0.05). Green dots are genes that are significantly upregulated in either Gcgr mAb or Gcgr−/− mice (p<0.05).

FIGS. 10A, 10B, 10C, and 10D show that alterations in lipid and bile acid metabolism and secreted factors in models of interrupted glucagon receptor signaling. Manhattan Plots of proteins significantly altered in serum of mice with (A) “acute” Gcgr mAb versus Gcgr+/+ PBS-treated mice and (B) “chronic” Gcgr−/− PBS-treated versus Gcgr+/+ PBS-treated mice Quantification of dose response to (B) Activin A and (C) hepcidin protein treatments on α-cell proliferation in cultured mouse islets treated for 3 days. **p<0.01 vs Gcgr−/− mouse serum-treated islets (red bar). (D) HPLC measurements of cholesterol, phospholipids, and triglycerides in serum after delipidation treatments.

FIGS. 11A, 11B, 11C, 11D, 11E, 11F, 11G, 11H, and 11I, show that high levels of serum L-glutamine and other amino acids in mice with interrupted glucagon signaling stimulate selective α-cell proliferation. (A) Quantification of L-alanine, L-citrulline, and L-ornithine dose response effects alone or in combination on α-cell proliferation in cultured mouse islets treated for 3 days. One-way ANOVA with Tukey posthoc analyses; ^{###}p<0.001 vs Gcgr−/− mouse serum-treated islets (red bar), ***p<0.001 vs control media-treated islets (first gray bar on left). (B) Quantification of total islet cell proliferation and (C) percentage of non α-cells proliferation in response to media with increasing amino acid concentration containing media for 3 days. White (low, n=3) to gray (intermediate, n=3-8) to black (high, n=7) color indicates concentration of total amino acids in each media condition. The red bar contains 10% Gcgr−/− serum (n=6) as in FIG. 1E. individual amino acid levels in cultured mouse islets treated for 3 days. One-way ANOVA with Tukey posthoc analyses. (D) Quantification of islet cell proliferation and (E) non-α-cell proliferation in response to altering individual amino acid levels in cultured mouse islets. One-way ANOVA with Tukey posthoc analyses; n=3, There were no statistical differences observed between high L-glutamate, L-leucine, and L-glutamine media (High ELQ)-treated islets, low L-glutamate, L-leucine, and L-glutamine media (Low ELQ)-treated islets, low L-glutamine (Low Q)-treated islets, low L-glutamate (Low E)-treated islets, or low L-leucine (Low L)-treated islets. (F) Quantification of rapamycin effects on amino acid stimulated islet cell and (G) non-α-cell proliferation in cultured mouse islets treated for 3 days. (H) Quantification of rapamycin effects on amino acid stimulated islet cell and (I) non-α-cell proliferation in cultured mouse islets treated for 3 days as a percentage of Ki67⁺ cells. One-way ANOVA with Tukey posthoc analyses; highest amino acid media with DMSO added (black bars n=3), highest amino acid media with 30 nM rapamycin added for the last 24 hours of culture (black left hashed bar, n=2), highest amino acid media with 30 nM rapamycin added for the full 72 hours of culture (black right hashed bar, n=3).

FIGS. 12A, 12B, 12C, 12D, 12E, 12F, 12G, 12H, 12I, 12J, 12K, 12L, 12M, and 12N show that human pancreatic islet α-cells proliferate when glucagon signaling is interrupted. (A) Perifusion of donor human islets used in these experiments. Insulin secretion in response to 5.6 mM to 16.7 mM glucose and 100 μM IBMX is shown in blue. (B) Donor human islet information. (C) Body mass over treatment (n=9-46), (D) random blood glucose over treatment (n=9-39), (E) glucose/arginine stimulated blood glucose (n=49-50), (F) glucose/arginine stimulated serum glucagon (n=49-50), and (G) glucose/arginine stimulated serum GLP-1 levels (n=10) in NSG mice treated with PBS (grey circles) or Gcgr mAb (grey triangles) for 14 days. (Student's t-test, ***p<0.0001; N.D. not detected). Note: Serum GLP-1 levels of all PBS-treated mice (n=10) were below the detection limit of the GLP-1 assay (41 pg/ml). The GLP-1 level for all glucose/arginine-stimulated Gcgr mAb mouse sample (n=10) were within the assay's detection limit. Therefore, no statistical analyses were performed. Representative images of α-cell proliferation in PBS and Gcgr mAb-treated mouse pancreas (H) Mouse pancreas mass (n=43-45) after 2 weeks of treatment with Gcgr mAb. (I-J) and human islet donor grafts (K-L) at 2 weeks treatment. Glucagon staining (green), Ki67 staining (red), and DAPI staining (blue) are shown. Proliferating α-cells (Ki67⁺ glucagom⁺) are indicated by white arrows. (M) Quantification of mouse α-cell proliferation (n=3) from non-responder human islet donors after 2 weeks of treatment with Gcgr mAb. Student's T-test analyses; *p<0.05 vs PBS-treated. (N) Human α-cell proliferation in donor islets pooled from 7 different experiments of NSG mice treated with PBS or Gcgr mAb and separated by responders (n=23) or non-responders (n=11). Student's T-test analyses; ***p<0.001 vs PBS-treated.

FIGS. 13A, 13A′, 13B, 13B′, 13C, 13D, 13D′, 13E, 13E′, 13F, 13F′, 13G, 13G′, 13H, 13H′, 13I, 13I′, 13J, 13J′, 13K, 13K′, 13L, 13L′, 13M, 13N, and 13O show mice with interrupted glucagon signaling (Gcgr^{Hep-/-}) had an 82-fold increase in the number of α-cells expressing SLC38A5 over control mice (Gcgr^{Hep|/|}).

FIGS. 14A, 14A′, 14B, 14B′, 14C, 14C′, 14D, 14D′, 14E, 14E′, 14F, 14F′, 14G, 14G′, 14H, 14H′, 14I, 14J, and 14K show α-cells in wildtype mice treated with GCGR mAb had upregulated expression of SLC38A5.

### DETAILED DESCRIPTION

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

A number of embodiments of the invention have been described. Nevertheless, it will be understood that various modifications may be made without departing from the spirit and scope of the invention. Accordingly, other embodiments are within the scope of the following claims.

Disclosed are improved compositions and methods for decreasing blood glucagon levels. As disclosed herein, L-glutamine and arginine are selective stimulators of α-cell proliferation generated when glucagon signaling is interrupted. Therefore, disclosed is a method for treating a subject with hyperglucagonemia, e.g., a subject with diabetes, that involves administering to the subject a composition comprising one or more L-glutamine inhibitors and/or one or more L-arginine inhibitors in an amount effective to decrease blood glucagon levels.

The L-glutamine and/or L-arginine inhibitors can in some cases be any agent capable of inhibiting an activity of L-glutamine and/or L-arginine. “Activities” of a protein include, for example, transcription, translation, intracellular translocation, secretion, phosphorylation by kinases, cleavage by proteases, homophilic and heterophilic binding to other proteins, and ubiquitination.

In some cases, the one or more L-glutamine inhibitor is a glutaminase (GLS) inhibitor. For example, the L-glutamine inhibitor can be Bis-2-(5-phenylacetamido-1,3,4-thiadiazol-2-yl)ethyl sulfide (BPTES). In some cases, the L-glutamine inhibitor comprises azaserine or 6-diazo-5-oxo-L-norleucine (L-DON). In some cases, the L-glutamine inhibitor comprises an inhibitor of a SLC7A5/SLC3A2 transporter. For example, the L-glutamine inhibitor can be 2-aminobicyclo-(2,2,1) heptanecarboxylic acid (BCH). In some cases, the L-glutamine inhibitor comprises an inhibitor of SLC1A5 transporter. For example, the L-glutamine inhibitor can be L-γ-glutamyl-p-nitroanilide (GPNA). In some cases, the L-glutamine inhibitor sequesters L-glutamine, e.g., removes L-glutamine from the circulation. For example, the L-glutamine inhibitor can be 4-Phenylbutyrate (4-PBA). In some cases, the L-glutamine inhibitor comprises an Asparaginase (such as, for example, Asparaginase Medac, Ciderolase, ONCASPAR®, ERWINASE®, ELSPAR®). Thus, in one aspect, disclosed herein are methods of treating a subject with hyperglucagonemia, e.g., a subject with diabetes, that involves administering to the subject a composition comprising one or more L-glutamine inhibitors; wherein the L-glutamine inhibitor is a glutaminase (GLS) inhibitor (such as, for example, Bis-2-(5-phenylacetamido-1,3,4-thiadiazol-2-yl)ethyl sulfide (BPTES); azaserine; 6-diazo-5-oxo-L-norleucine (L-DON); an inhibitor of a SLC7A5/SLC3A2 transporter (such as, for example, 2-aminobicyclo-(2,2,1) heptanecarboxylic acid (BCH)); an inhibitor of SLC1A5 transporter (such as, for example, L-γ-glutamyl-p-nitroanilide (GPNA)); 4-Phenylbutyrate (4-PBA) and/or an Asparaginase (such as, for example, Asparaginase Medac, Ciderolase, ONCASPAR®, ERWINASE®, ELSPAR®).

In some cases, the one or more arginine inhibitor is a nitric oxide synthase (NOS including, but not limited to the three NOS isoforms endothelium, neuronal and inducible), dimethylarginine dimethylaminohydrolase (DDAH), and arginase. Thus, in one aspect, disclosed herein are methods of treating a subject with hyperglucagonemia, e.g., a subject with diabetes, that involves administering to the subject a composition comprising one or more L-arginine inhibitors; wherein the one or more L-arganine inhibitors comprises NOS, DDAH, or arginase.

The disclosed methods can be used to treat any disease or condition characterized by hyperglucagonemia. In some cases, the subject has pre-diabetes, type 1 diabetes, type 2 diabetes, or gestational diabetes. In some cases, the subject has an A1C≧5.7, fasting glucagon level≧55 pM, fasting blood glucose≧100 mg/dl, two hour glucose tolerance test level of ≧140 mg/dL, or any combination thereof.

The disclosed methods can further involve co-administering one or more additional therapeutic agents for treating diabetes, which can be in the same composition or in separate compositions. For example, the one or more L-glutamine inhibitors and/or one or more arginine inhibitors can administered in combination with metaformin or insulin. In some cases, the L-glutamine inhibitor and/or arginine inhibitor is administered in combination with a glucagon-like peptide 1 (GLP-1) agonist, DDP-4 inhibitor, or combination thereof.

Also disclosed is a method for identifying a pancreatic-alpha-cell proliferation inducing compound. This method can involve contacting at least one pancreatic alpha-cell with a given compound, and testing whether said compound is capable of ki67 or pHH3 gene or protein expression. In some cases, the method involves contacting at least one pancreatic alpha-cell with a given compound, and testing whether said compound is capable Edu or BrdU incorporation.

Also disclosed is a method for expanding alpha cells in culture that involves contacting the alpha cells with an effective amount of a composition comprising L-glutamine. The method can further involve transdifferentiating the expanded alpha cells into beta cells. For example, this can involve the use of GABA agonists, positive allosteric modulators, Arx siRNA inhibitor, antisense oligos, or ribozymes, or Pax4 overexpression. U.S. patent publication US20080318908 A1 and International patent publications WO2014048788 A1 and WO 2006015853 A3 are incorporated by reference for the teachings of these methods. This method can further involve transplanting the beta cells into a subject with diabetes. In some embodiments, the alpha cells are autologous.

Also disclosed are beta cells produced by transdifferentiation of alpha cells according to the disclosed methods that can be used for transplantation. Also disclosed are kits containing either L-glutamine for transdifferentiating alpha cells, or beta cells produced from transdifferentiation, in combination with an immunosuppressive agent. For example, the immunosuppressive agent can be selected from the group consisting of azathioprine, mycophenolic acid, leflunomide, teriflunomide, methotrexate, FKBP/cyclophilin, tacrolimus, ciclosporin, pimecrolimus, abetimus, gusperimus, sirolimus, deforolimus and everolimus.

### EXAMPLES

### Example 1

### Interruption of Hepatic Glucagon Signaling Reveals a Hepatic-Islet α-Cell Axis where L-Glutamine Stimulates α-Cell Proliferation

α-Cells Proliferate in Response to a Hepatic Factor in Serum

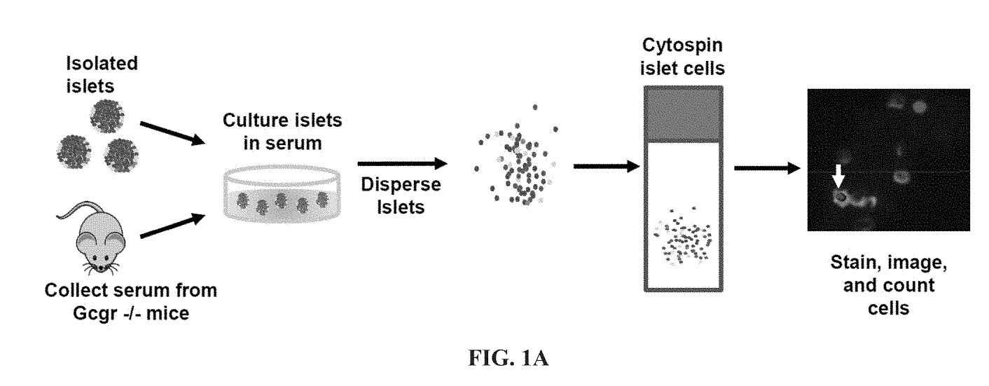

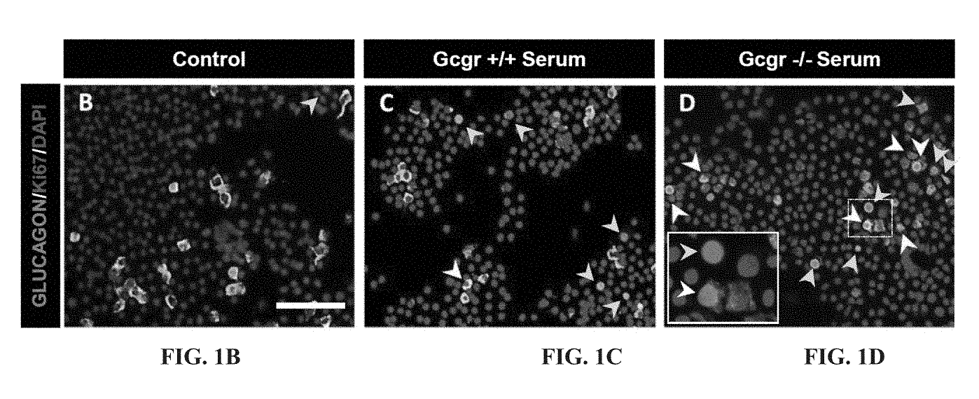

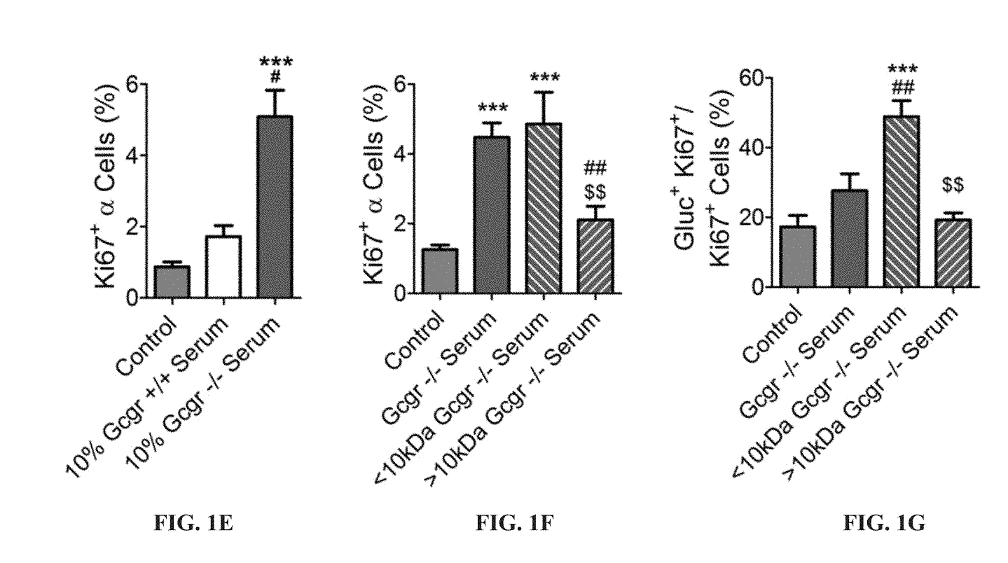

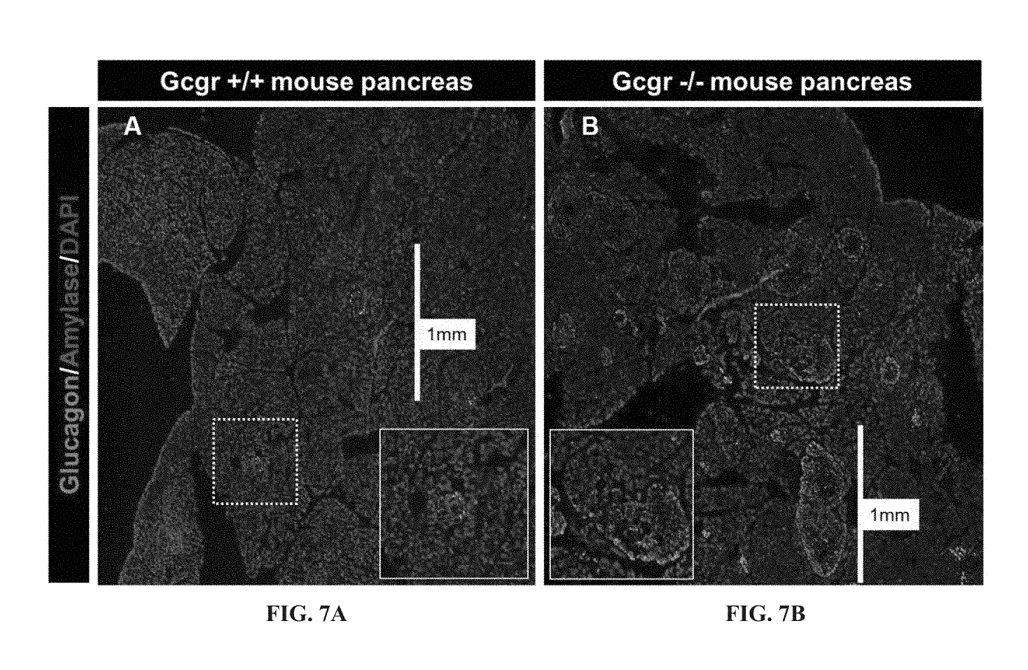

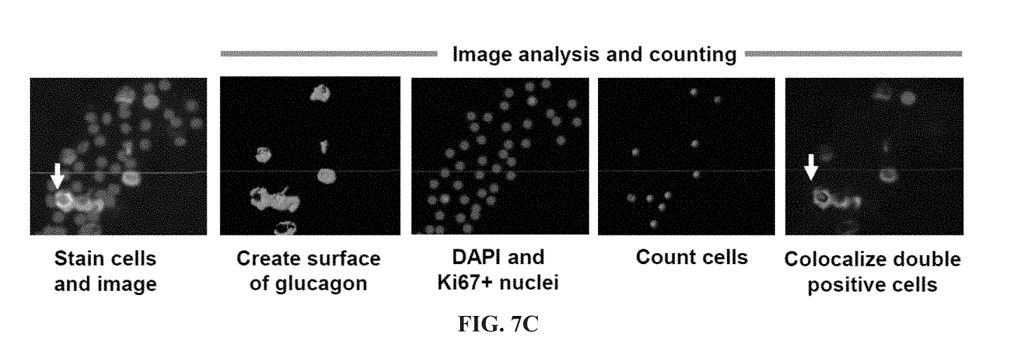

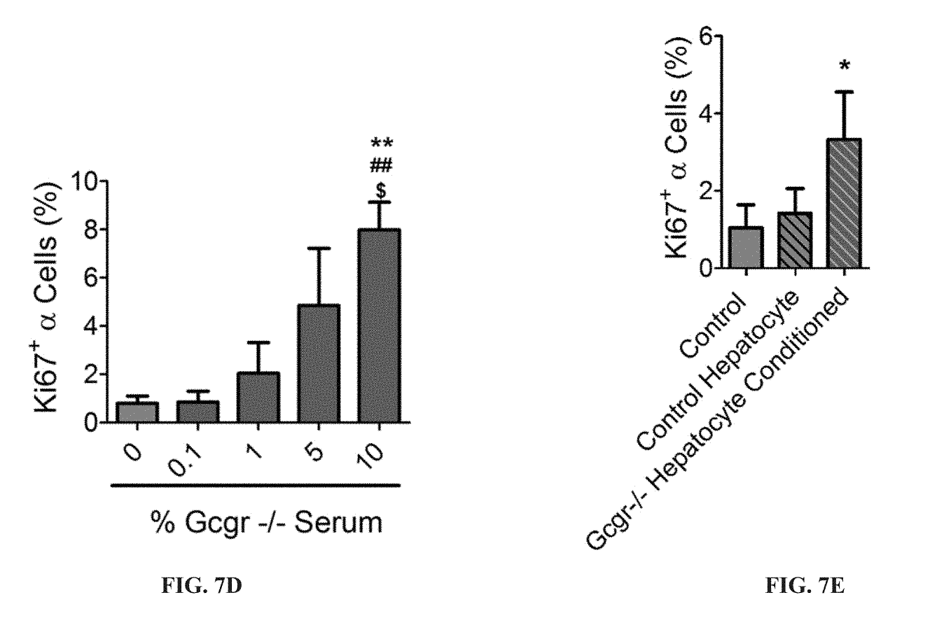

Mice with interrupted glucagon signaling (Gcgr−/−) develop α-cell hyperplasia (FIG. 7A,B). To investigate whether the serum of these mice contained a factor that stimulates α-cell proliferation, a new in vitro assay was developed to assess α-cell proliferation. Challenges for in vitro α-cell proliferation assays include that the number of α-cells in an islet is quite low (<15% of islet cells), the α-cell proliferation rate in islets is quite low (<2%), and baseline proliferation rates of α-cell lines are too great to use to identify a serum mitogen. To maximize the ability to quantify proliferating α-cells intact mouse islets were first cultured in media supplemented with Gcgr−/− or Gcgr+/+ mouse serum, dispersing the islets into cells, and then the cells centrifuged into a focused monolayer of cells using a cytospin centrifuge (FIG. 1A). This allowed thousands of α-cells in each sample to be counted, requiring far fewer islets with no need for sectioning as used in traditional islet embedding technique that capture only a few α-cells in each section. To further maximize the throughput of this assay, a cytonuclear algorithm was developed to automate quantification of α-cell proliferation in cytospin islet images (FIG. 7C). Culturing wildtype islets in media containing 10% Gcgr−/− mouse serum for 3 days increased α-cell proliferation 3 fold versus culture with 10% Gcgr+/+ mouse serum or control media alone (FIG. 1B and FIG. 7D).

To test the hypothesis that the liver was the source of this signal, pancreatic islets were treated with conditioned media from cultured Gcgr−/− mouse hepatocytes. Gcgr−/− mouse hepatocyte conditioned media stimulated α-cell proliferation suggesting that liver produces an α-cell stimulating factor (FIG. 7E).

To characterize the serum factor(s), Gcgr−/− mouse serum was size fractionated. There was a <10 kDa fraction stimulated α-cell proliferation 4 fold, similar to unfractionated Gcgr−/− serum (FIG. 1C). The >10 kDa fraction of Gcgr−/− serum did not stimulate α-cell proliferation. Also analyzed was what proportion of cells proliferating under each condition were α-cells. While the total number of proliferating islet cells (including α-cells, β-cells, δ-cells, endothelial cells, etc.) was lower in the <10 kDa Gcgr−/− serum fraction, the total α-cell proliferation rate was the same as unfractionated Gcgr−/− serum. Thus, the fractionation of Gcgr−/− serum process partially purified an α-cell selective mitogen(s) with approximately half of islet proliferating cells being α-cells with <10 kDa fractionated Gcgr−/− serum treatment versus <20% with unfractionated Gcgr−/− serum treatment (FIG. 1D). Together, these data indicate that α-cell proliferation in response to interrupted glucagon signaling is likely due to a small serum protein/peptide or small molecule (e.g. lipid, amino acid, or metabolite).

The mTOR Signaling Pathway is Critical Mediator of α-Cell Proliferation in Islets

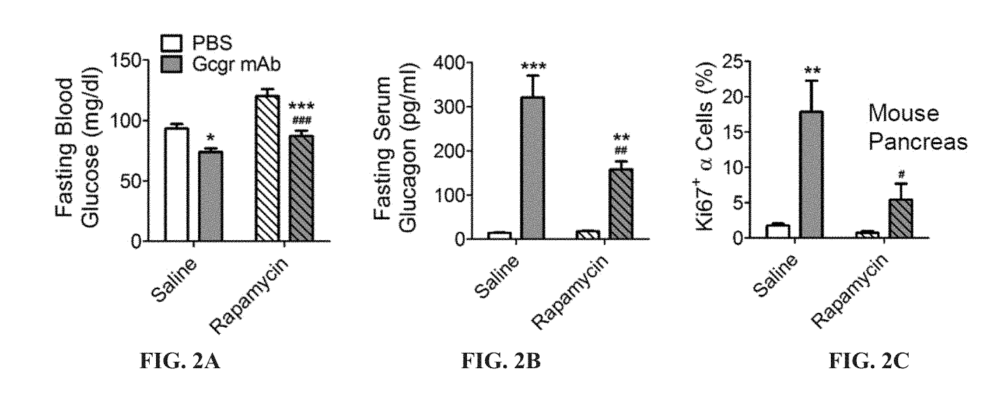

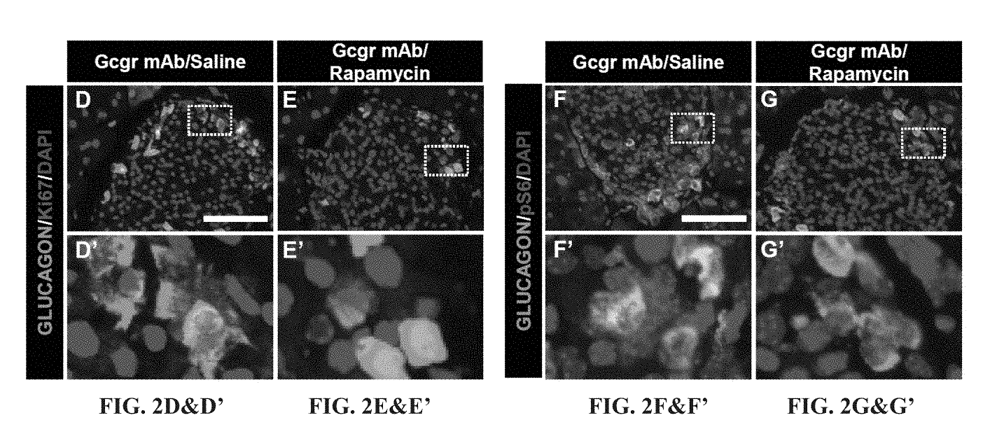

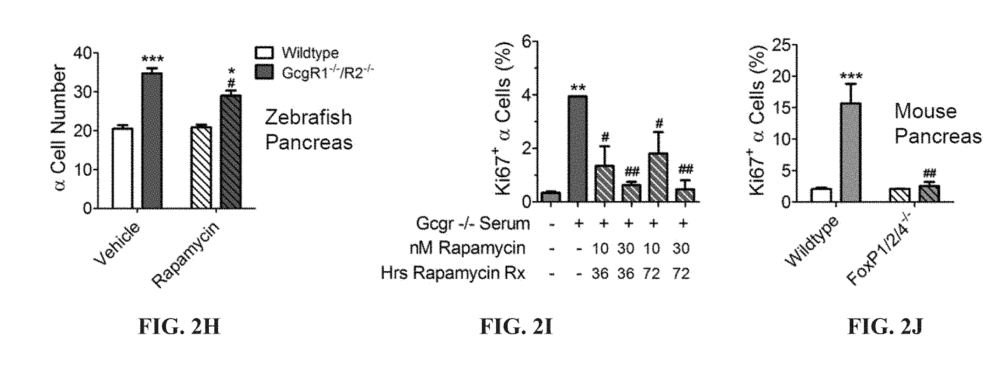

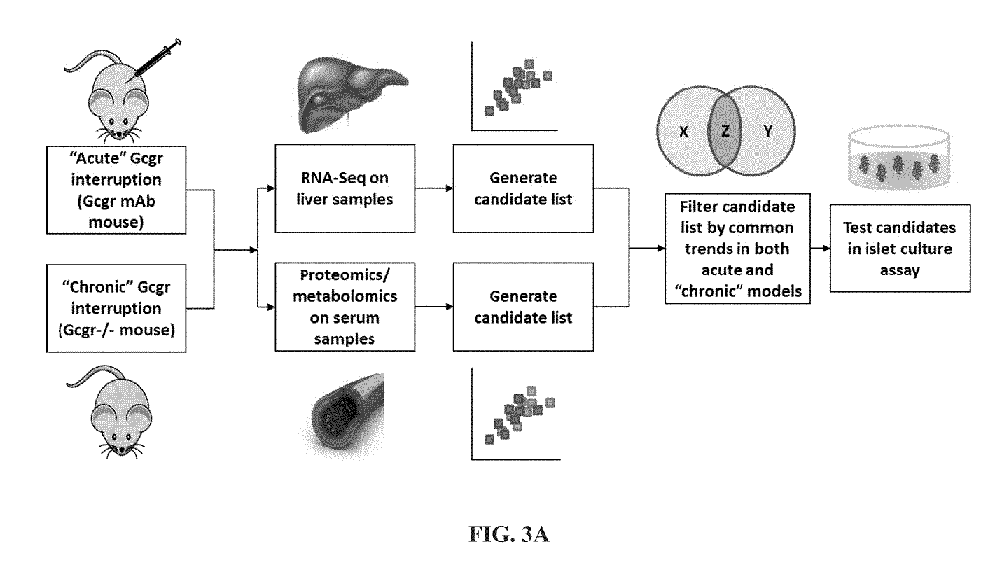

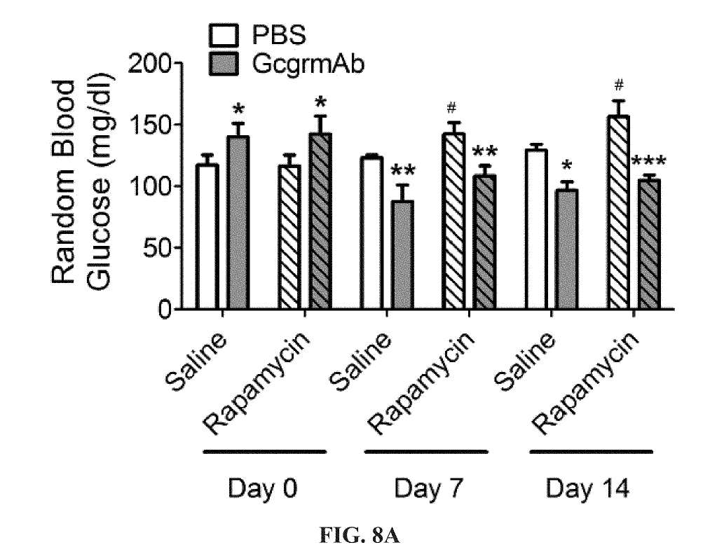

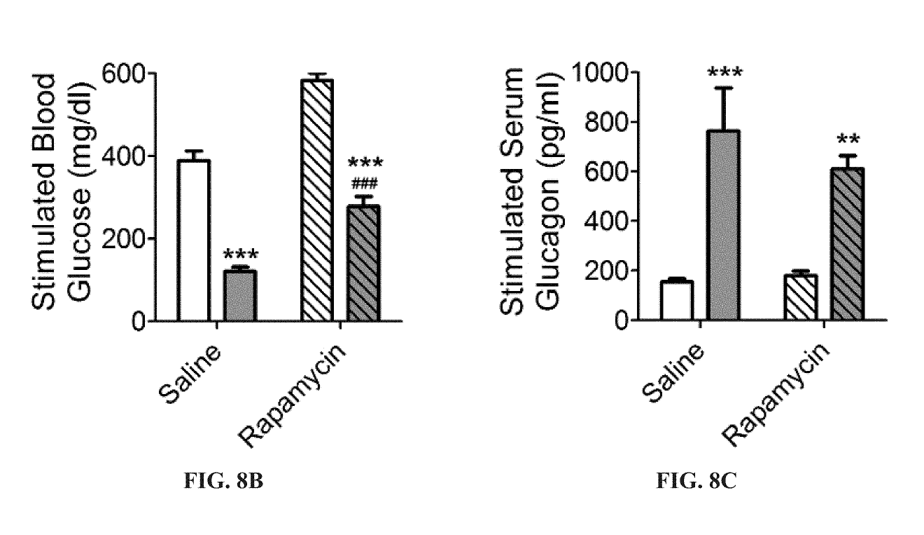

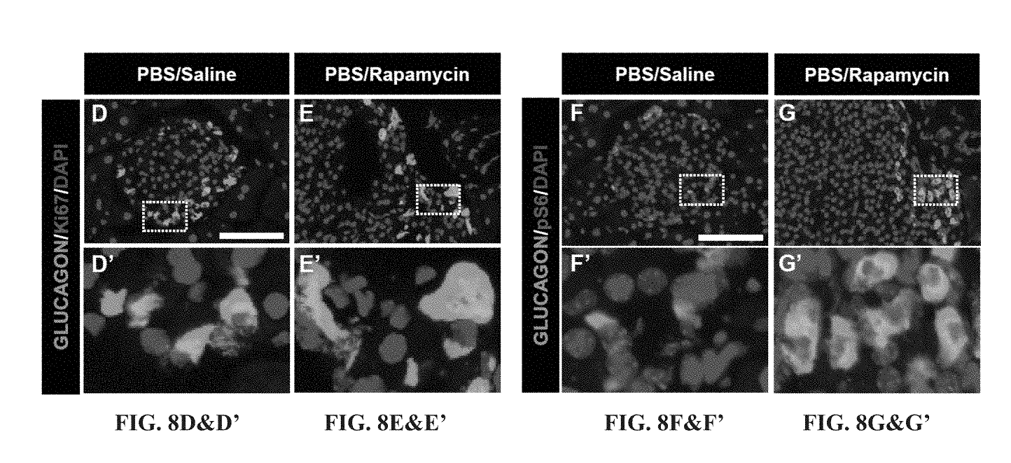

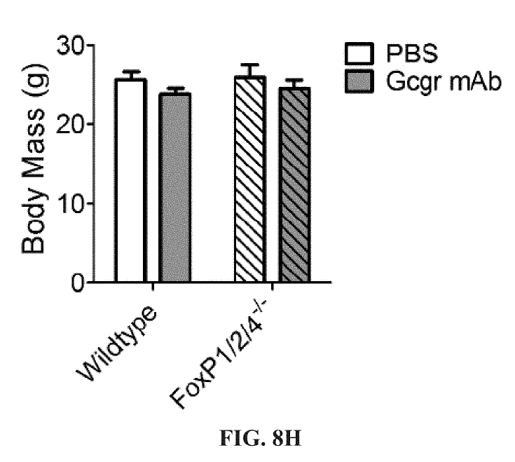

Interrupting glucagon signaling in mice for as little as 6 weeks using a monoclonal antibody against Gcgr (Gcgr mAb) results in α-cell hyperplasia. Treatment for 2 weeks lowers blood glucose, robustly increases serum glucagon levels, and stimulates α-cell proliferation (FIGS. 2A-D and 8A-D), suggesting that interruption of glucagon signaling is rapidly generating this signal. To understand the signaling pathways required for the increased α-cell proliferation, the mTOR kinase pathway was examined, which integrates multiple signaling pathways (e.g. growth factor and nutrient sensing) to stimulate cell proliferation, metabolism, and macromolecule synthesis. There was activation of a downstream target of mTOR kinase, S6 protein, by robust pS6 (p235/236) colocalization in the peri-islet region of the pancreas and within α-cells and δ-cells of Gcgr mAb-treated mice whereas pS6 staining in α-cells of PBS-treated mice was very rare (FIGS. 2F, 8F). To directly test the role of mTOR signaling in α-cell proliferation, mice were cotreated with Gcgr mAb and rapamycin, a potent inhibitor of mTOR kinase. Rapamycin treatment partially suppressed the hyperglucagonemia and α-cell proliferation induced by Gcgr mAb administration similar to the reduction observed in Gcgr−/− mice (Solloway et al., 2015; FIG. 2B-E). Rapamycin also largely attenuated the α-cell expression of pS6 (p235/236) in Gcgr mAb cotreated mice (FIGS. 2F-G and 8F-G).

A zebrafish model of interrupted glucagon signaling that that develops hyperglucagonemia and α-cell hyperplasia was also used to further test the role of mTOR signaling in α-cell proliferation. As in the mouse model, treatment of zebrafish larvae with rapamycin reduced the number of α-cells induced by interruption of glucagon signaling (FIG. 2H).

Since systemic treatment with rapamycin could potentially block production, release, or action of the α-cell mitogen, experiments were conducted to determine whether activation of mTOR signaling was required for α-cell proliferation in the in vitro α-cell proliferation assay. Mouse islets cultured with Gcgr−/− mouse serum in the presence of rapamycin blocked the proliferative effects of Gcgr−/− mouse serum (FIG. 21). These data indicate that mTOR signaling in the islet is required for the α-cell factor in Gcgr−/− serum to stimulate α-cell proliferation.

Fox P Transcription Factors are Required for α-Cell Proliferation in Response to Interrupted Glucagon Signaling

FoxP transcription factors are critical for α-cell mass expansion observed during early postnatal development. To test whether FoxP signaling has a role in the α-cell in response to interrupted glucagon signaling, FoxP1/2/4 islet null mice were treated with Gcgr mAb. While wildtype Fox P mice had a robust increase in α-cell proliferation, FoxP1/2/4 islet null mice had reduced α-cell proliferation (FIG. 2J). Thus, FoxP signaling in α-cells is required for expansion of α-cells when glucagon signaling is blocked.

Liver Transcriptomics and Serum Proteomics/Metabolomics Analyses Reveal Alterations in Serum Proteins and Lipid and Amino Acid Metabolism

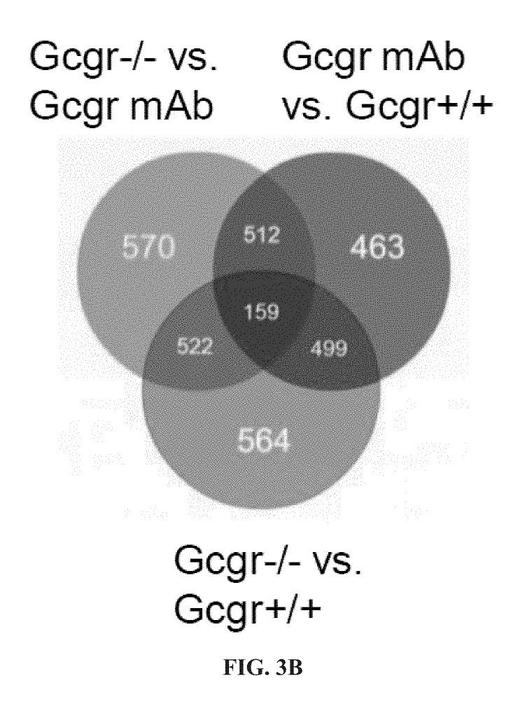

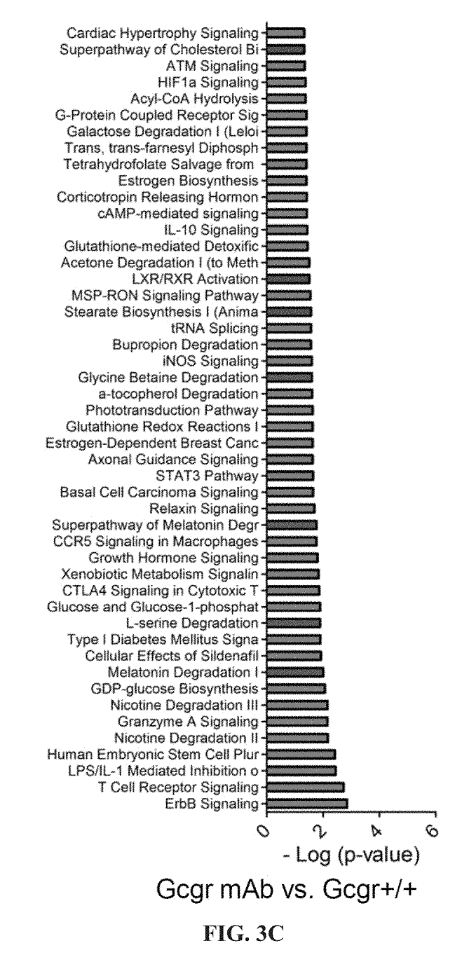

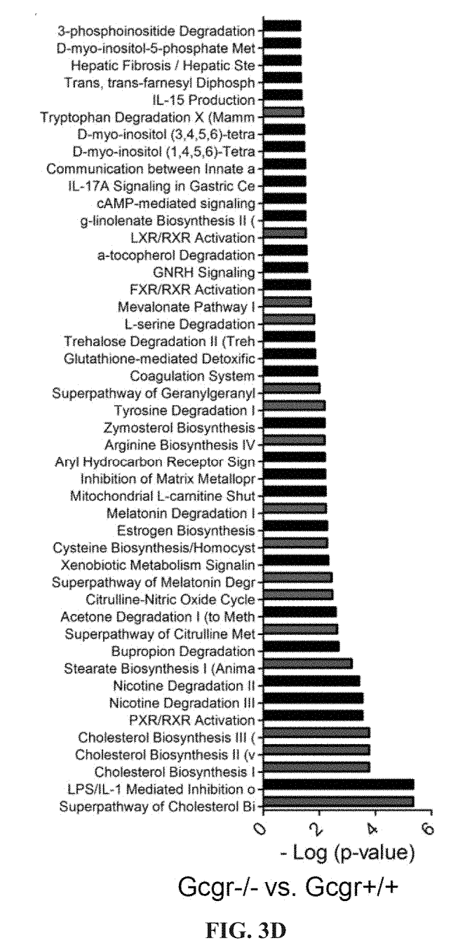

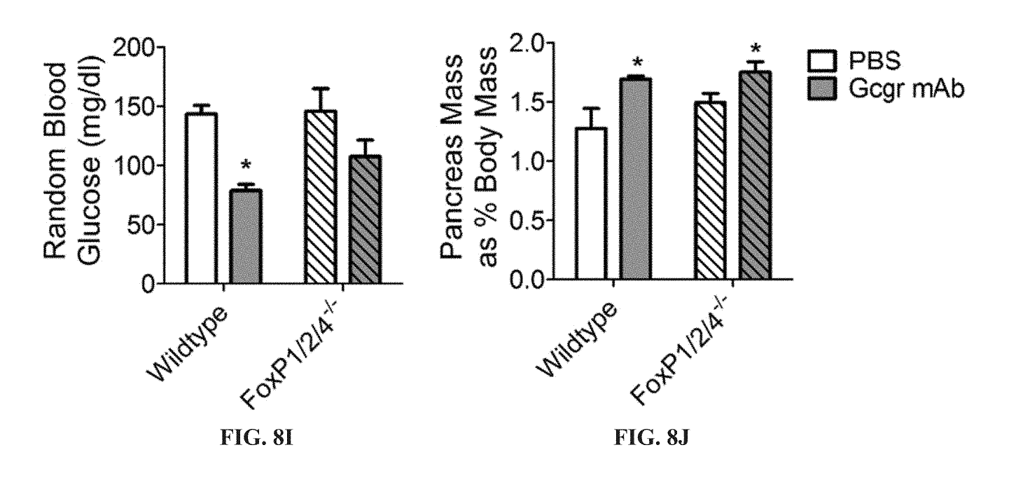

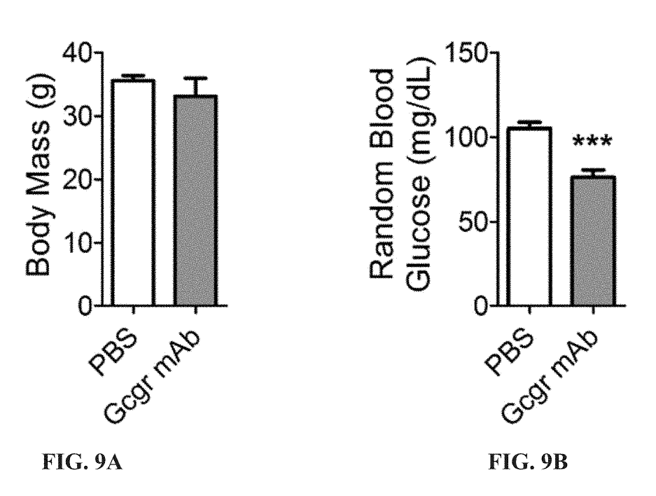

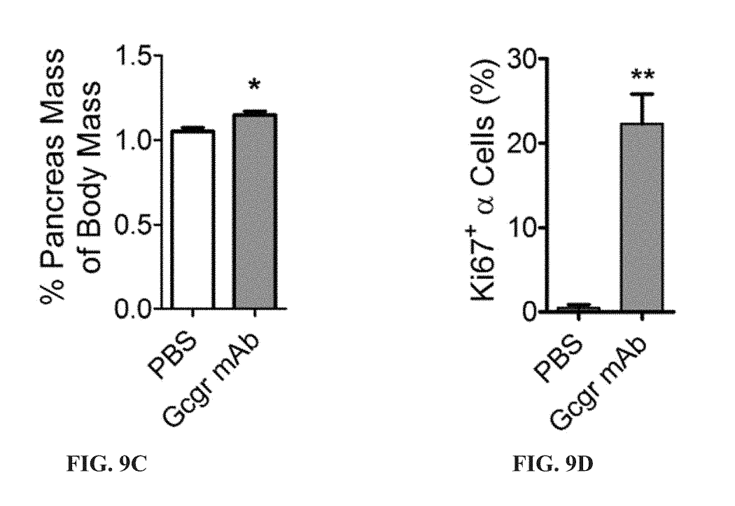

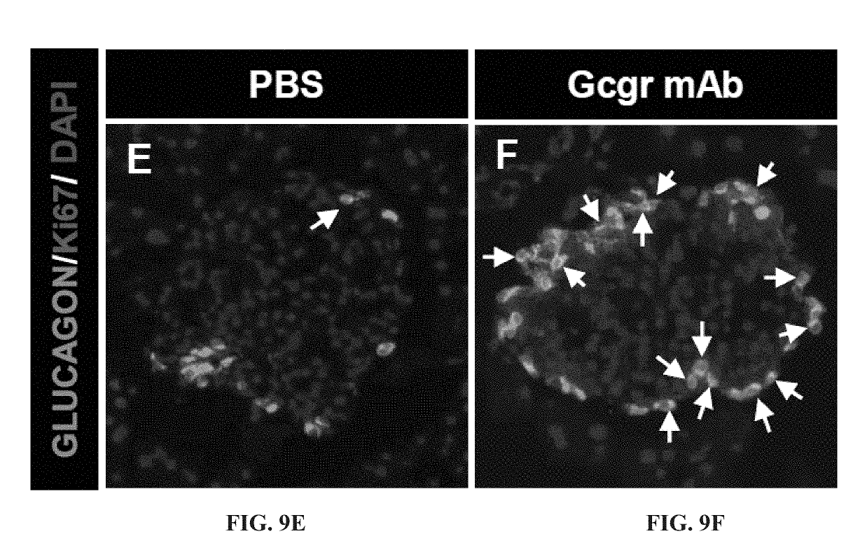

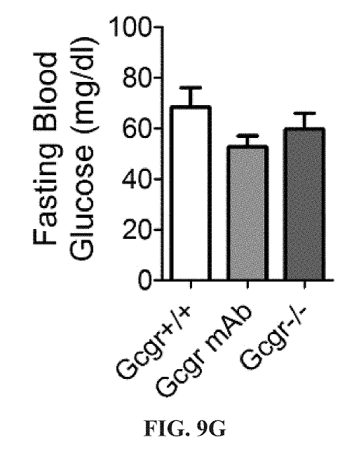

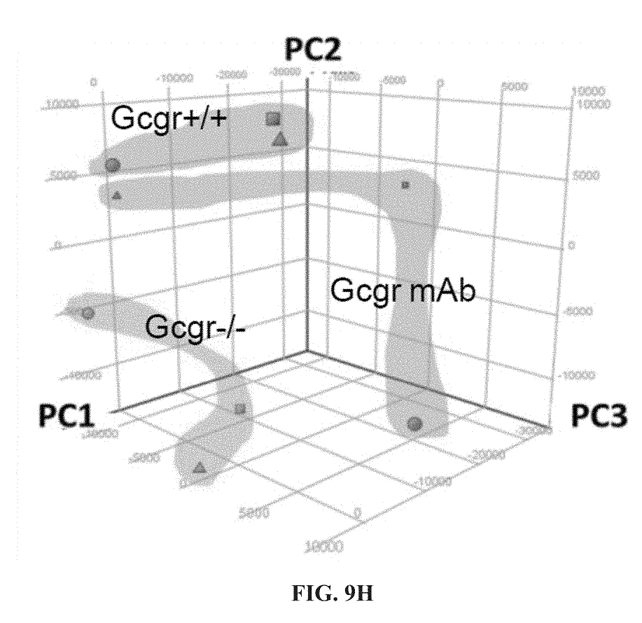

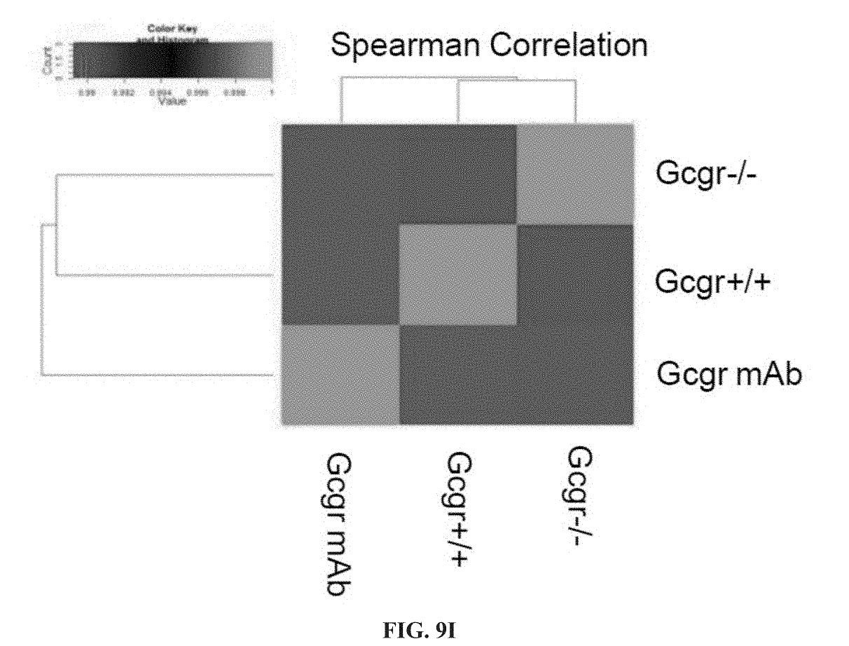

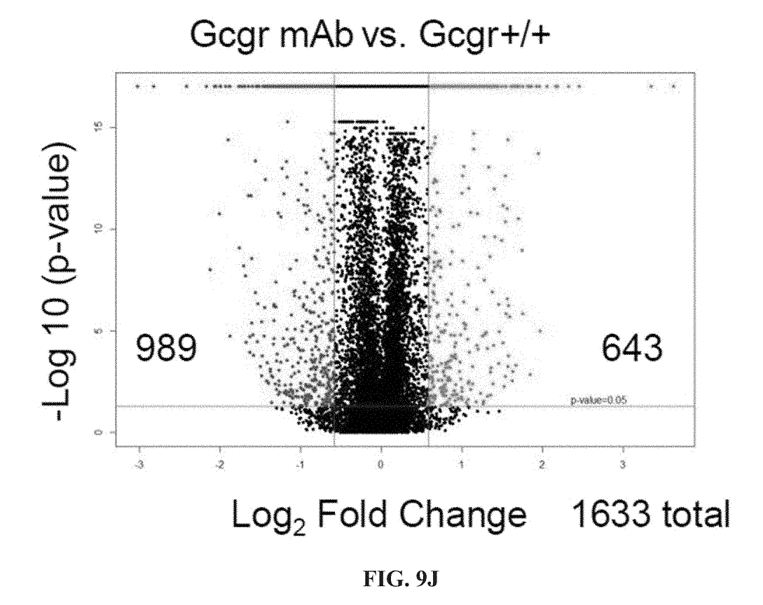

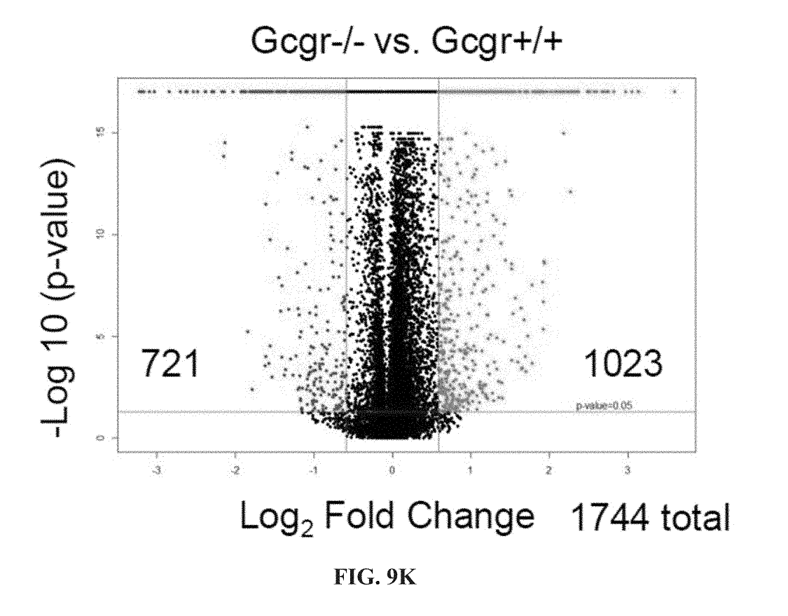

Based on the size fractionation studies, the factor stimulating α-cell proliferation could have been a small macromolecule, peptide, or non-protein small molecule derived from the liver. To generate a candidate list to test in the in vitro proliferation assay, both a “chronic” model of interrupted glucagon signaling (Gcgr−/−) and an “acute” model of interrupted glucagon signaling (Gcgr mAb-treated) were leveraged and compared since both models have increased α-cell proliferation (FIG. 3A). Gcgr−/− mice have increased α-cell proliferation as early as 6 weeks of age. Ten days of Gcgr mAb treatment increased α-cell proliferation rates approximately 14-fold (FIG. 9E-F).

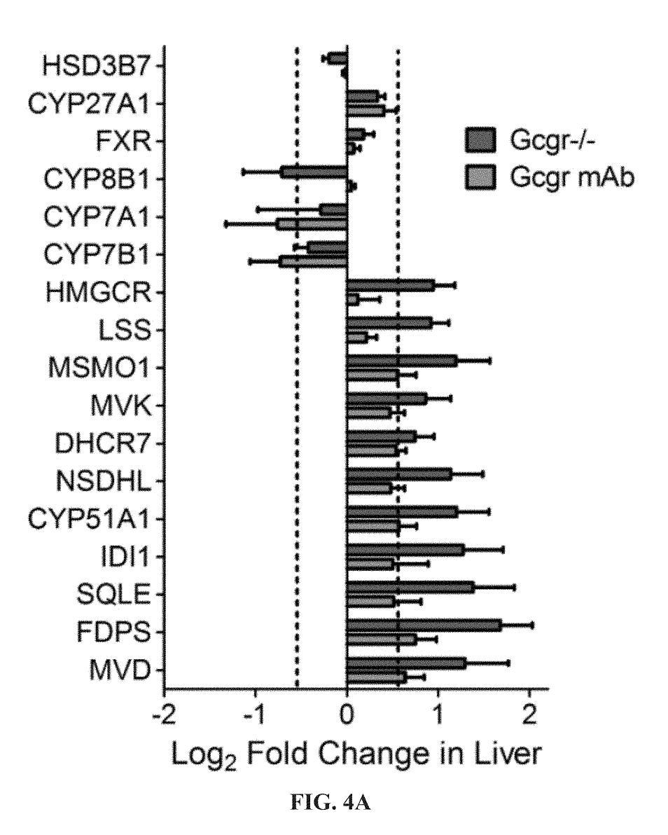

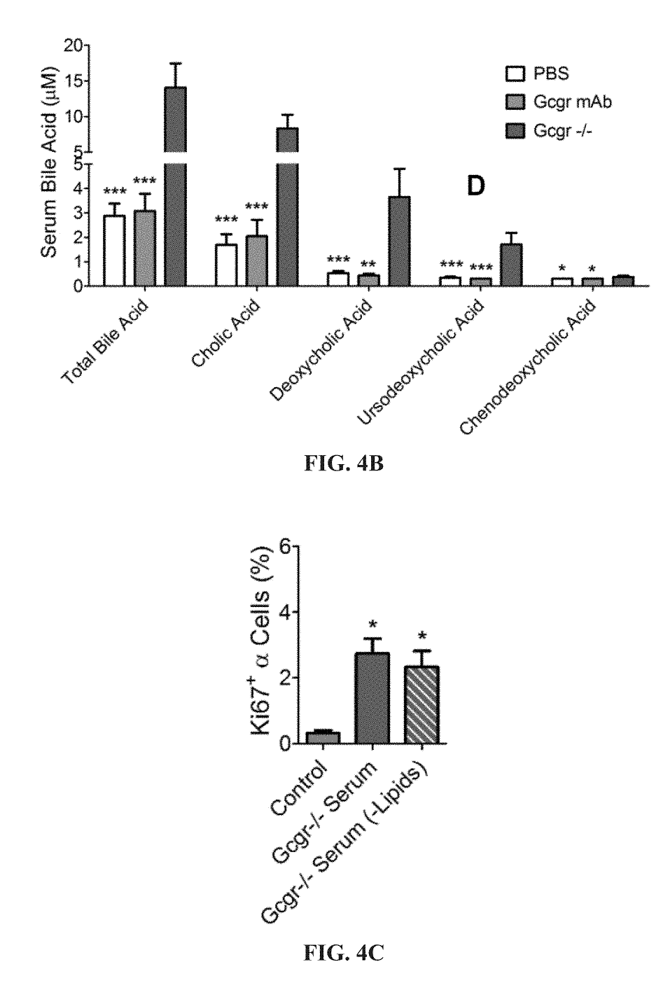

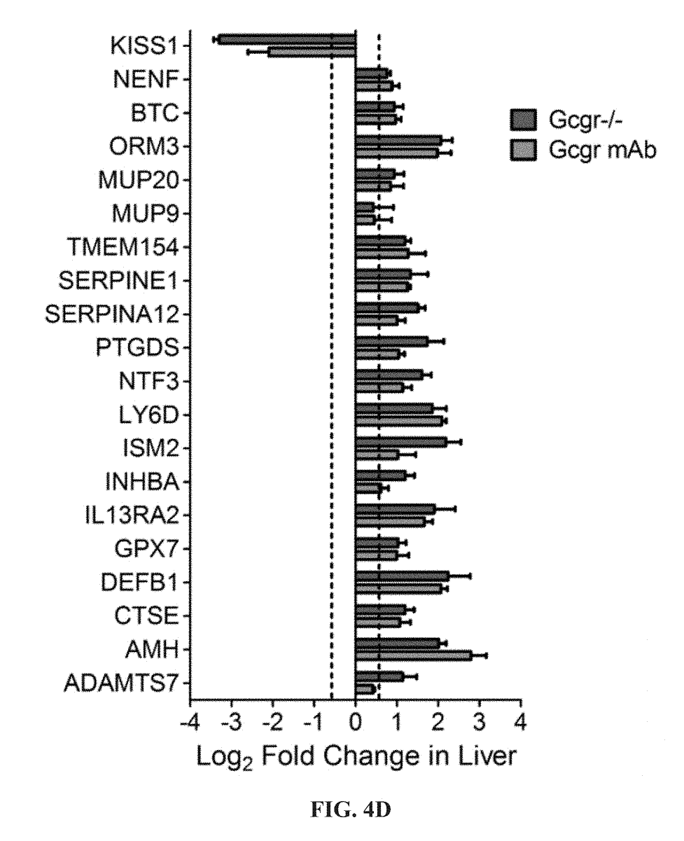

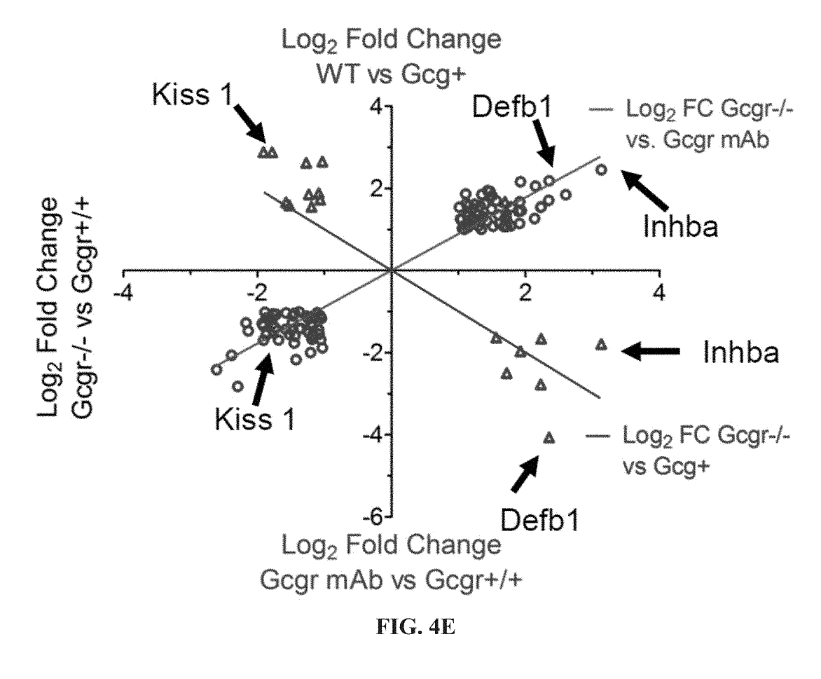

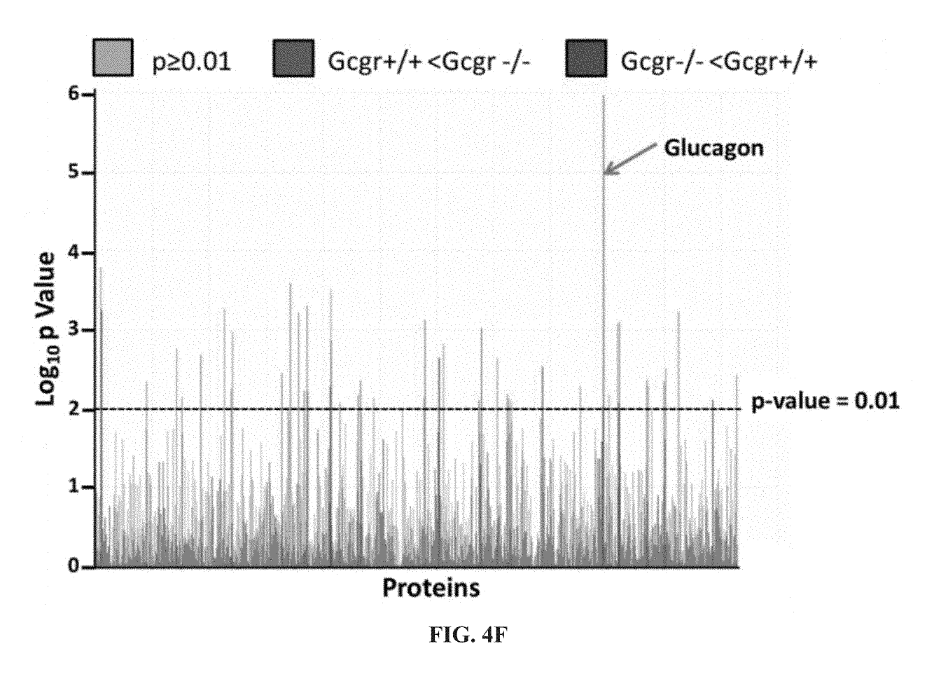

The hepatic transcriptional profile from mice with acute and chronic interruption of glucagon signaling were compared. 658 genes were either upregulated or downregulated (FIGS. 9J-K). The initial focus was on genes predicted to encode secreted factors by analyzing the predicted protein sequence for canonical or non-canonical secretion motif. Of the predicted secreted proteins, some (Inhba-activin A, Defb1-defensin b1, and Hamp1-hepcidin) are less than 10 kDa in size (FIG. 4B). One secreted factor, Kisspeptin (Kiss1) was strongly downregulated in both “acute” and “chronic” interruption of glucagon secretion. Kiss1 was identified in transcriptomic analyses as a gene strongly upregulated in response to glucagon signaling and may be responsible for impaired insulin secretion in response to hyperglucagonemia. Since Kiss1 expression data was in accordance with predictions that interruption of glucagon signaling would downregulate Kiss1 gene expression, top gene expression changes were cross-referenced with expression data generated by Song et al. to identify which genes most strongly regulated either positively or negatively by glucagon. Therefore, Log 2 fold change gene expression in a model of enhanced glucagon signaling (WT vs. Gcg+) from Song et al. and gene expression data from the “chronic” model of interrupted glucagon signaling (Gcgr+/+ vs. Gcgr−/−) were compared to determine if those genes strongly upregulated by interruption of glucagon signaling are also strongly downregulated by enhancement if glucagon signaling (open blue triangles) and vice versa. The top 107 genes that are most significantly altered in both models of interrupted glucagon signaling, 19 were significantly altered by enhanced glucagon signaling (FIG. 4C). Of the 7 genes that were upregulated in interrupted and downregulated in enhanced glucagon signaling, two genes (Inhba and Defb1) produce proteins known to be small peptides (activin A and defensin b1).

To complement discovery efforts to identified altered hepatic expression of secreted factors, both aptamer-based proteomics were performed on whole serum and LC-MS/MS on fractionated serum from mice with interrupted glucagon signaling. It was reasoned that this could identify factors that are both upregulated in hepatic transcriptome analyses and serum proteomic analyses. 66 proteins were identified in the serum of mice. Despite size fractionation, LC-MS/MS analyses of <10 kDa Gcgr−/− serum revealed considerable contamination of a 54 kDa protein band that we confirmed to be albumin. This was removed by antibody-targeted albumin-depletion columns. LC-MS/MS analyses on albumin-depleted <10 kDa Gcgr−/− serum and <10 kDa Gcgr+/+ serum found only 7 peptides unique to <10 kDa Gcgr−/− serum corresponding to 4 large proteins, α1 antitrypsin, keratin, haptoglobin, and polymeric immunoglobulin receptor. One of these α1 antitrypsin (SerpinA1) is a high molecular weight highly abundant protein (HAP). When highly abundant proteins from Gcgr−/− serum were removed with antibody-targeted HAP depletion columns or treated fractionated serum with proteases (e.g. trypsin and proteinase K), the <10 kDa Gcgr−/− serum fraction retained its ability to stimulate α-cell proliferation. Therefore, if the factor were proteinaceous it would be protease-resistant, too small to be a HAP, and low in concentration making detection by LC-MS/MS techniques difficult.

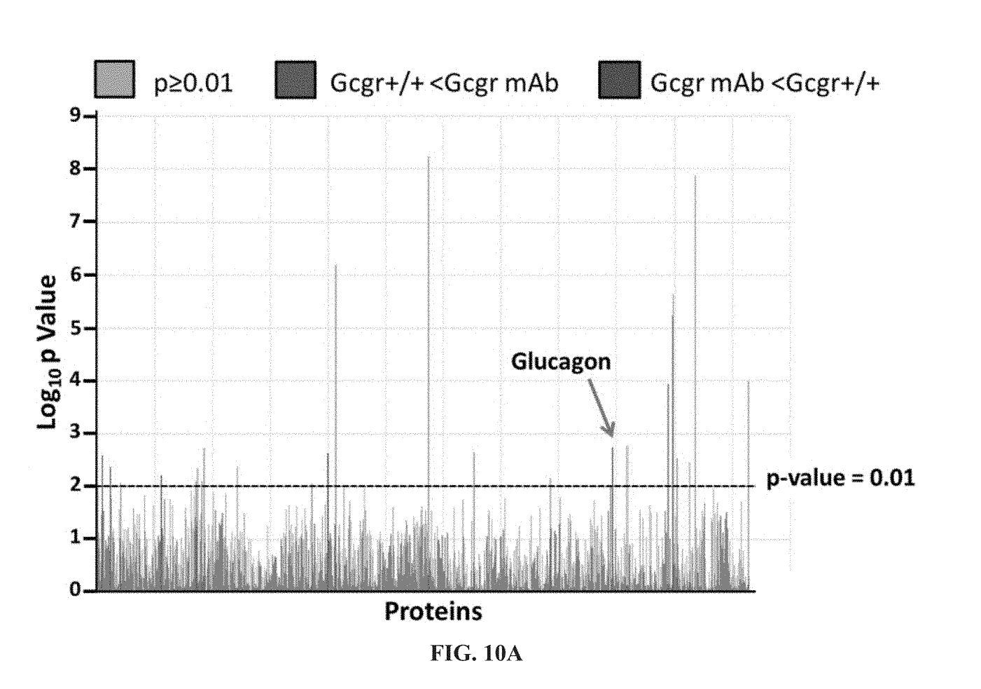

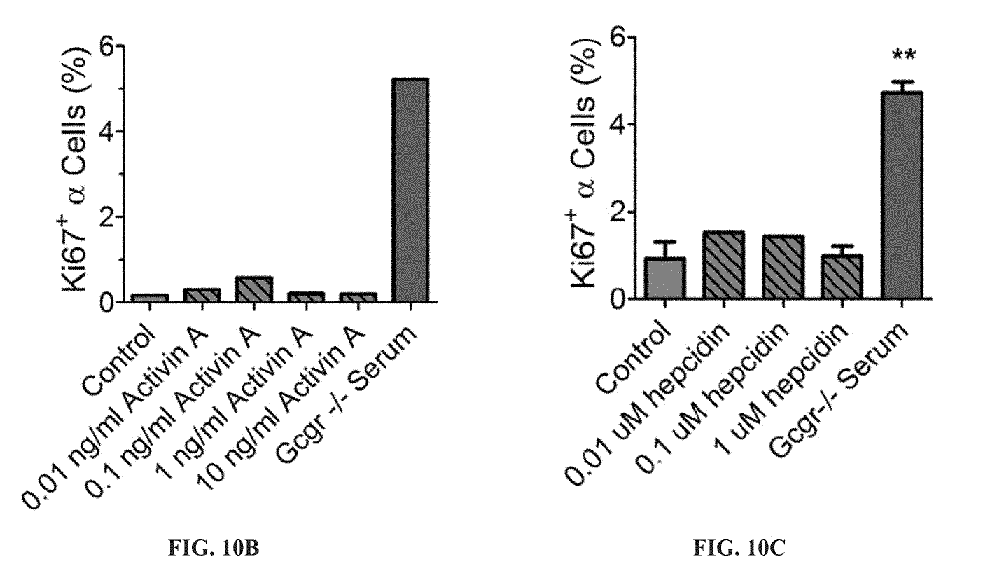

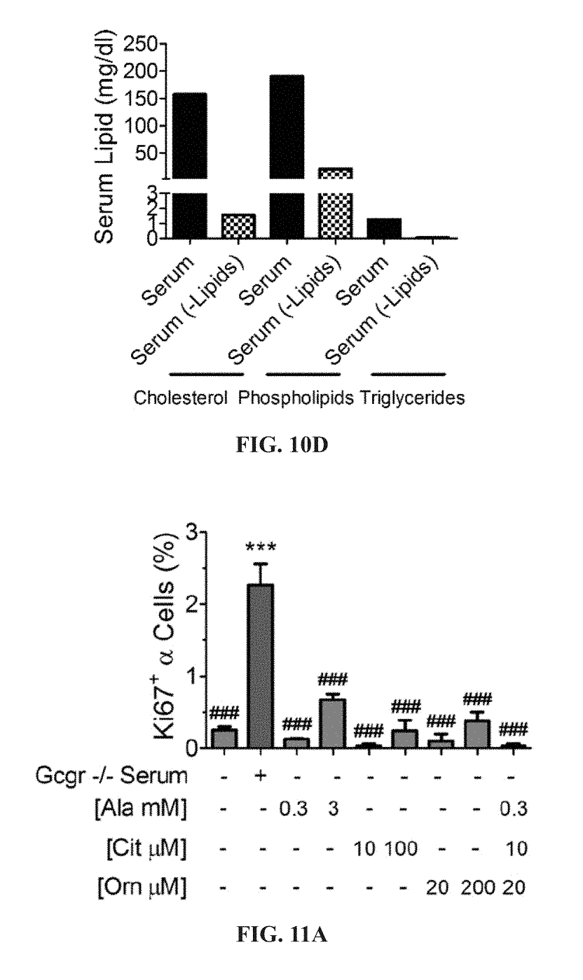

Over 1200 serum proteins were analyzed by aptamer-based screening. Nine proteins (glucagon, BMP-1, BGH3/Tgfbi, activin A, notch 1, WIF-1, TF/thromboplastin, VEGF sR3, and testican-2) were upregulated in both Gcgr−/− and Gcgr mAb mice (FIGS. 4F and 10A). Glucagon was significantly upregulated in both models validating that this method could detect hyperglucagonemia known to occur in both models. Therefore, proteomic analyses revealed proteins that are upregulated in the serum of mice with interrupted glucagon secretion; however, only one of these (Inhba/activin A) was upregulated transcriptionally in the liver of both models as well.

IPA© analyses revealed alterations in expression of genes related to canonical pathways involved in both lipid/cholesterol and amino acid metabolism (FIGS. 3C-D). Since these analyses suggested alterations in lipid metabolism and glucagon signaling impacts cholesterol biosynthesis, hepatic lipid oxidation and accumulation, expression of genes involved in lipid and cholesterol metabolism were analyzed. Both models of mice with interrupted glucagon signaling had increased expression of genes involved in lipid and cholesterol biosynthesis (FIG. 4A). However, Gcgr−/− and Gcgr mAb-treated mice did not upregulate expression of genes regulating bile acid synthesis and transport (Cyp7a1, Cyp7b1, Cyp8b1, and Slc10a1) (FIG. 4A). Interestingly, while synthesis was unlikely to be increasing in either model, bile acid levels were upregulated in the serum Gcgr−/− mice, but not in Gcgr mAb-treated mice (FIG. 4B), excluding bile acids as the factor(s) stimulating α-cell proliferation.

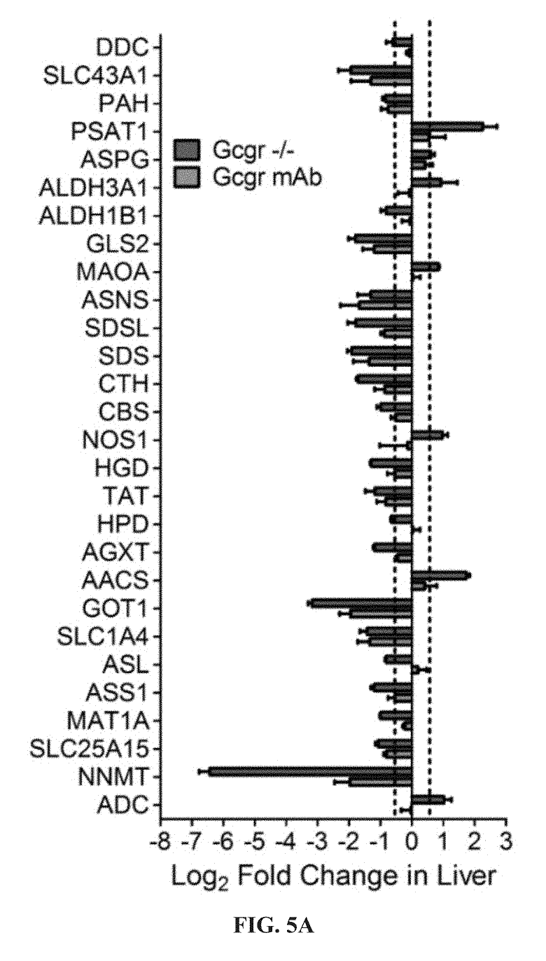

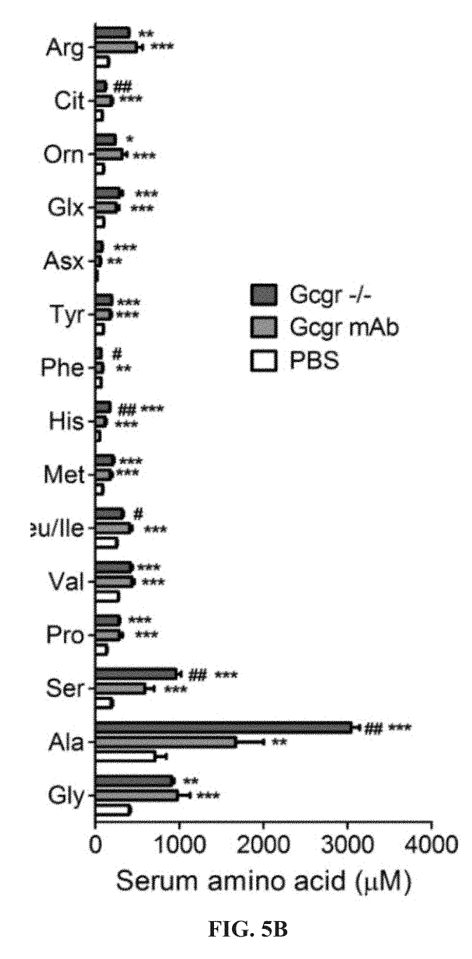

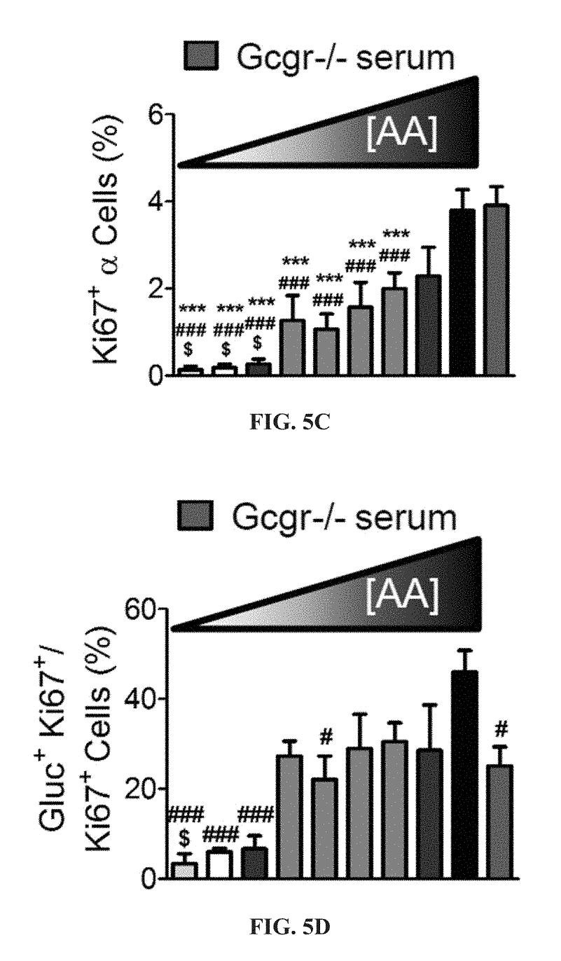

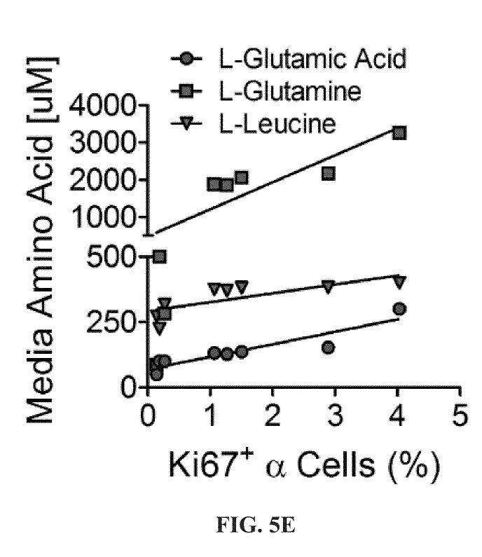

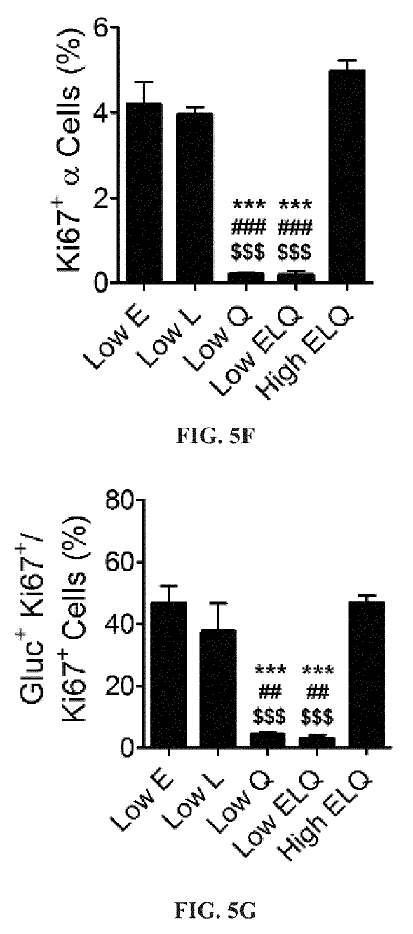

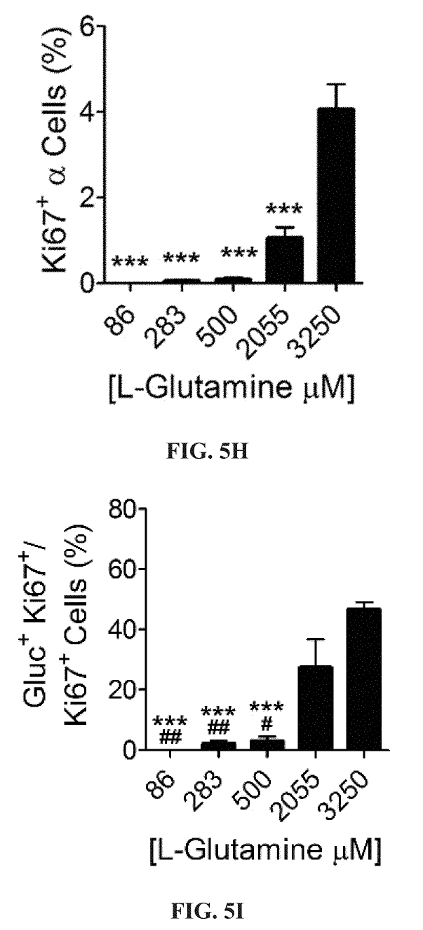

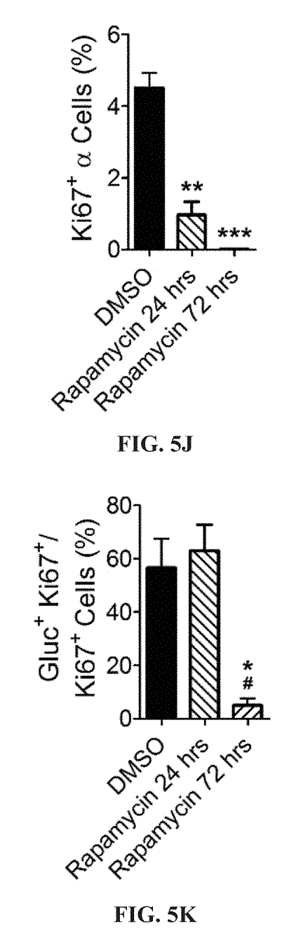

In addition to alterations in lipid metabolism, there were changes in gene expression that encode proteins involved in hepatic amino acid metabolism (FIGS. 3C-D and 5A). Because the changes in gene expression predicted impaired catabolism of most amino acids in the liver, serum amino acid levels were analyzed in mice with both “acute” and “chronic” interruption of glucagon signaling. All major serum amino acids except phenylalanine were significantly elevated 1.2-5 fold (FIG. 5B). Together, this strategy of hepatic transcriptional profiling coupled with serum fractionation and proteomic/metabolomic analyses identified candidate factors for the increased α-cell proliferation when glucagon signaling is interrupted.

Candidate Testing Reveals that Amino Acids Potently Stimulate α-Cell Proliferation

The candidate factors identified through the systems biology approach were next tested using the in vitro α-cell proliferation assay. In testing whether lipids in Gcgr−/− serum could stimulate α-cell proliferation, it was determined that serum activity was retained after the removal of >99% of triglycerides, cholesterols, and phospholipids (FIG. 4D). Of the secreted proteins, activin A was a top candidate as it was upregulated in both models and in both transcriptomics and proteomic analyses. However, it did not stimulate α-cell proliferation in vitro (FIG. 10C). Furthermore, treating Gcgr−/− serum with proteases did not block α-cell proliferation in cultured islets. Together, these studies indicate that the hepatic factor is a small, non-lipid molecule.

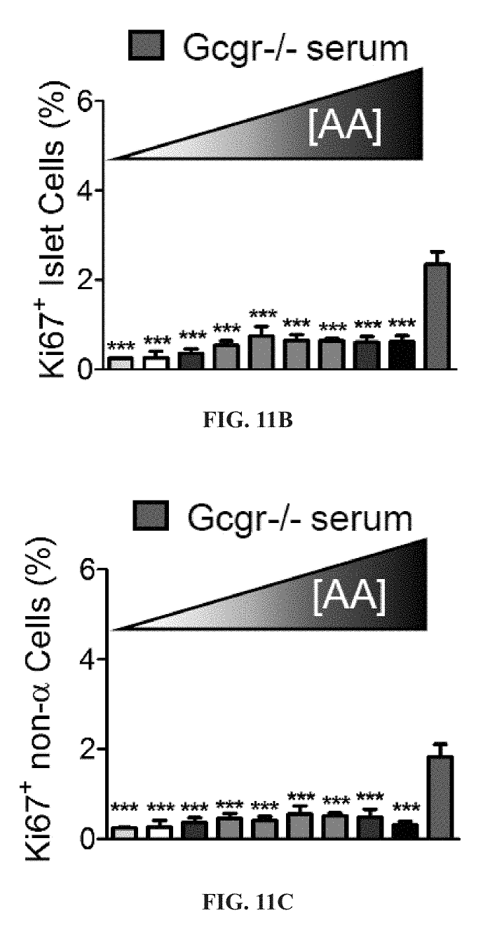

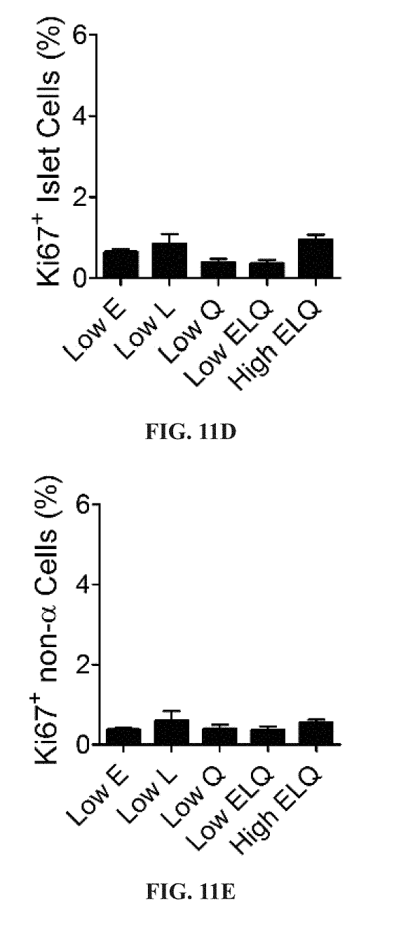

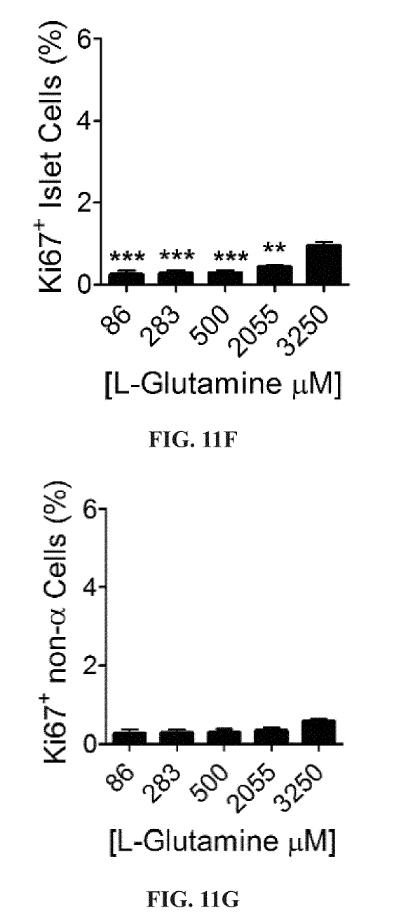

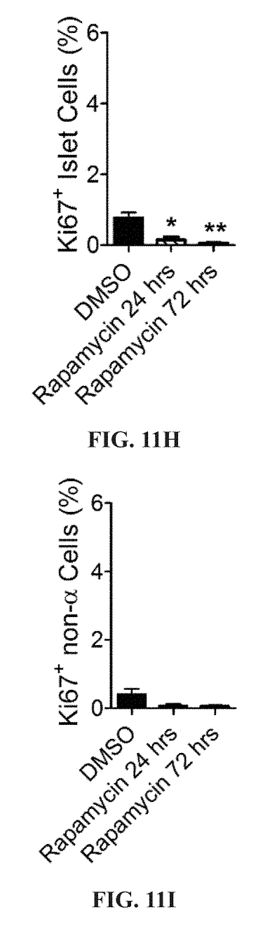

Since some amino acids (e.g. arginine) stimulate glucagon release, 23 different amino acids were tested in combination or alone at doses spanning concentrations found in both Gcgr+/+ and Gcgr−/− serum. First, amino acids that were elevated in Gcgr−/− mouse serum but not present in RPMI media were tested, reasoning that by adding Gcgr-−/− serum to culture media, a factor not found in culture media could be added. Citrulline, ornithine, and alanine alone or in combination failed to stimulate α-cell proliferation at concentrations observed in Gcgr−/− serum (FIG. 11A). To test whether elevated amino acids collectively could stimulate α-cell proliferation in vitro, media was prepared to recapitulate the amino acid concentrations in Gcgr+/+ or Gcgr−/− mouse serum. Media containing high levels of amino acids found in whole (100%) Gcgr−/− serum (black bar), but not the levels found in whole (100%) Gcgr+/+ serum (white bar), potently stimulated α-cell proliferation in our islet culture assay and was similar to 10% whole Gcgr−/− mouse serum-supplemented media (red bar) (FIG. 5C). Additionally, when media conditions recapitulated only amino acids concentrations found in 10% Gcgr−/− serum treated cultures (dark gray bar), there was no difference in the α-cell proliferation rate when compared to 10% whole Gcgr−/− mouse serum-supplemented media (red bar), suggesting that whatever factors present in mouse serum could be mimicked simply by addition of amino acids (FIG. 5D). Interestingly, media amino acids at all concentrations tested had no effect on non α-cell proliferation rates resulting in the majority of proliferating cells in the islet cultures being α-cells under high amino acid conditions (FIGS. 5C and 5E). These data suggest that amino acids at levels found in Gcgr−/− serum are sufficient to selectively stimulate α-cell proliferation.

L-Glutamine Selectively Stimulates α-Cell Proliferation in Pancreatic Islets

To determine which amino acid(s) stimulated α-cell proliferation, we prepared media having higher total levels (darker gray bars) of amino acids and lower total levels (lighter gray bars) of amino acids (FIG. 5C and Table 1). Islets cultured in media with the highest total amino acid concentrations (black bar) had the greatest α-cell proliferation rate while islets cultured in media with the lowest total amino acid concentrations (white bar) had the lowest α-cell proliferation rate (FIG. 5C). Rapamycin blocked increased α-cell proliferation in response to high amino acid containing media (FIG. 5J). As the total amino acid concentration increased in the culture media the α-cell proliferation rate also increased. In the few exceptions where total levels of amino acids were high but did not stimulate α-cell proliferation, there were a few individual amino acids that had lower concentrations than what were found in Gcgr−/− serum. By lowering only a few amino acids at a time, linear regression analyses was performed on each amino acid concentration versus the α-cell proliferation achieved by that media. 3 amino acids [L-leucine (L), L-glutamic acid (E), and L-glutamine (Q)] had concentrations significantly and positively correlated with increased α-cell proliferation (FIG. 5G). Experiments were conducted to determine whether high levels of each of these three amino acids were required for the increased α-cell proliferation observed in high amino acid containing media treatment by reducing the concentration of each to Gcgr+/+ serum levels (Low) while maintaining high concentrations of all other amino acids (FIG. 5G). Reduction of all three of these amino acids together (Low ELQ) resulted in a complete loss of α-cell proliferation compared to media with Gcgr−/− serum levels of each amino acid (High ELQ) (FIG. 5H). Individually, lowering neither L-leucine (Low L) nor L-glutamic acid (Low E) alone to levels in Gcgr+/+ serum levels had an effect on α-cell proliferation in the presence of high amino acid containing media. Therefore, high levels of L-leucine and L-glutamate or not required for increased α-cell proliferation in response to high amino acid levels. However, lowering L-glutamine (Low Q) to levels observed in Gcgr+/+ serum levels abolished the stimulation of α-cell proliferation in the presence of high amino acid containing media (FIG. 5G). L-glutamine concentration had no effect on non-a islet cell proliferation (FIG. 11C). Furthermore, L-glutamine in a dose-dependent fashion increased α-cell proliferation in the presence of high amino acids (100% of Gcgr−/− serum mimicking levels) (FIG. 5I). Together, these data indicate that L-glutamine is the factor that stimulates α-cell proliferation in mice with interrupted glucagon signaling.

Human Islet α-Cells Proliferate in Response to Interrupted Glucagon Signaling

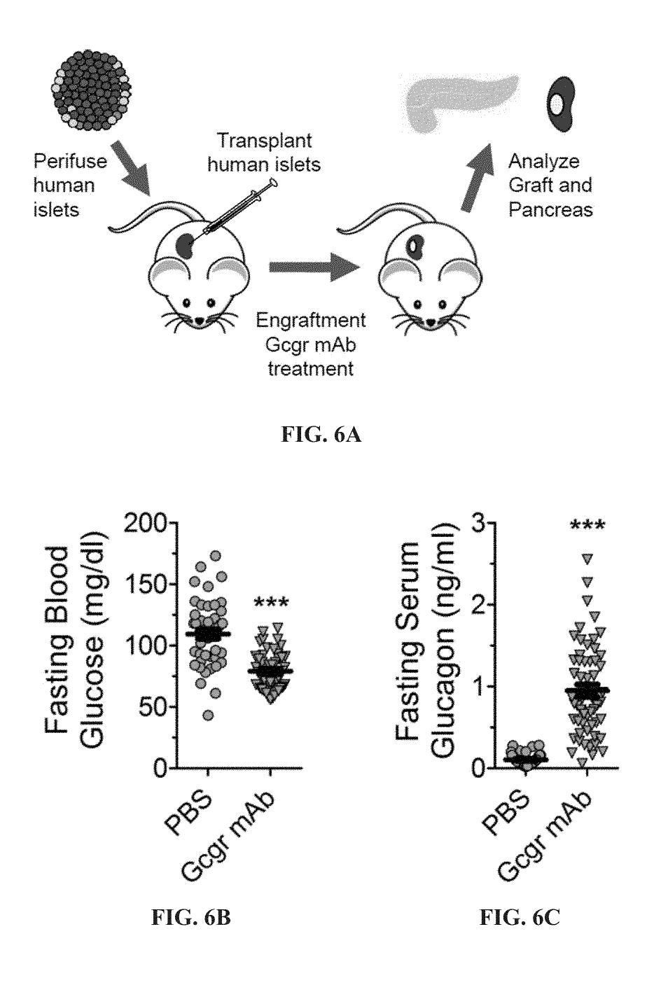

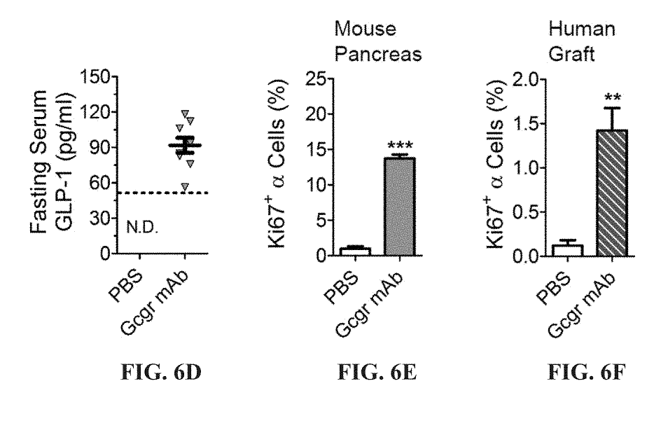

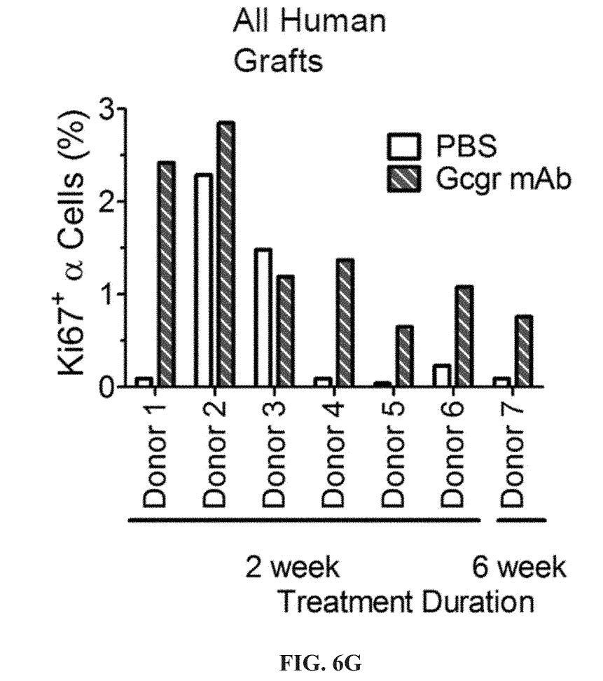

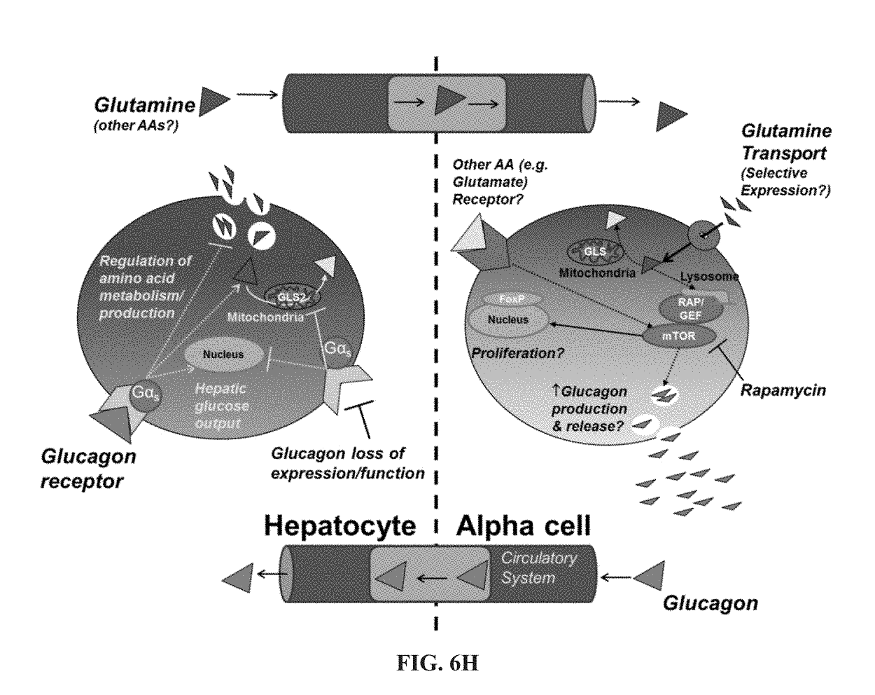

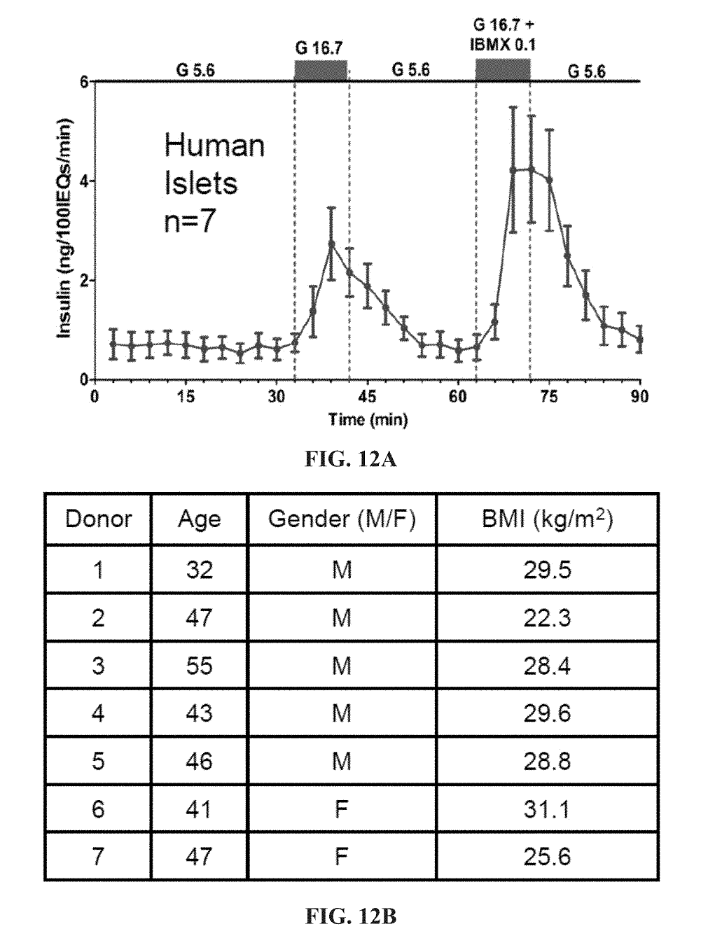

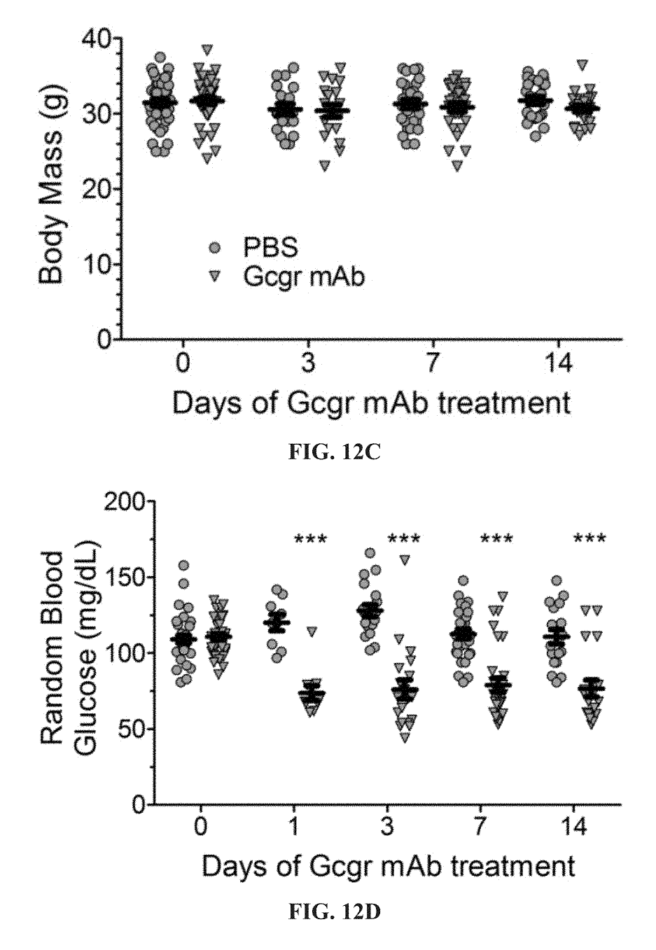

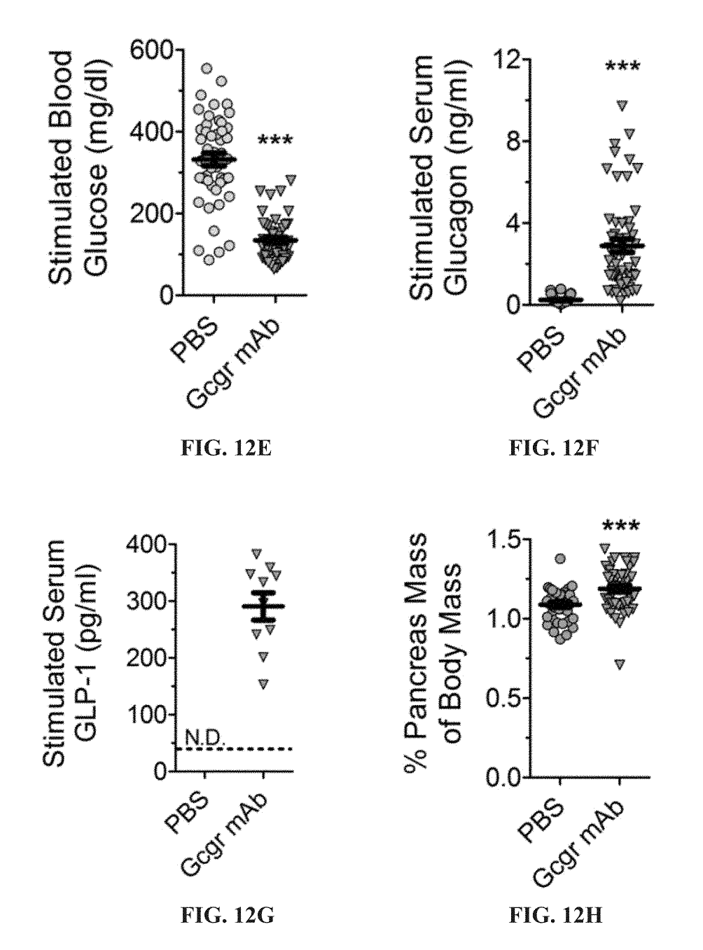

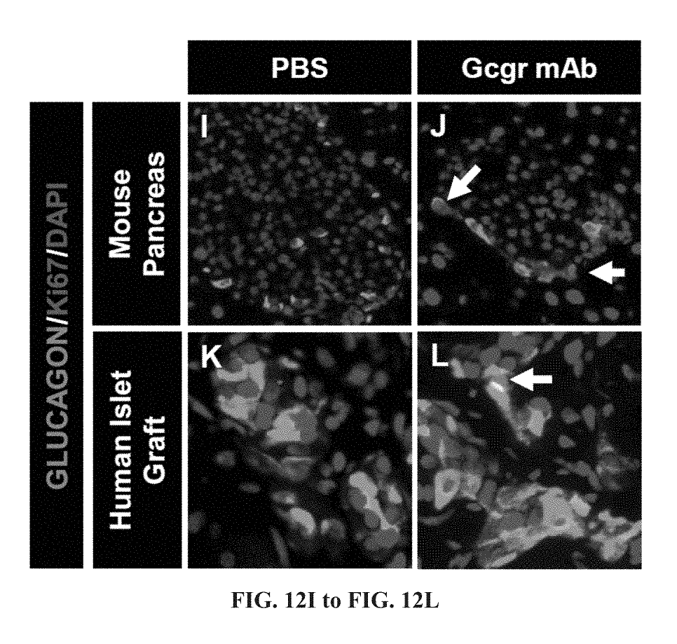

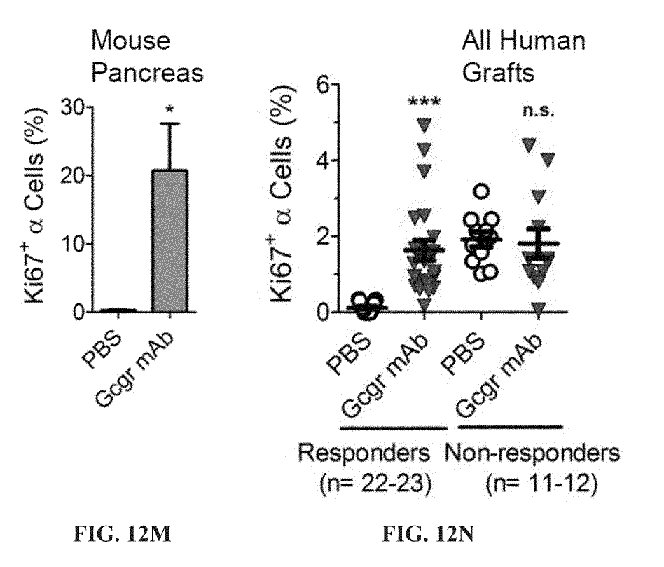

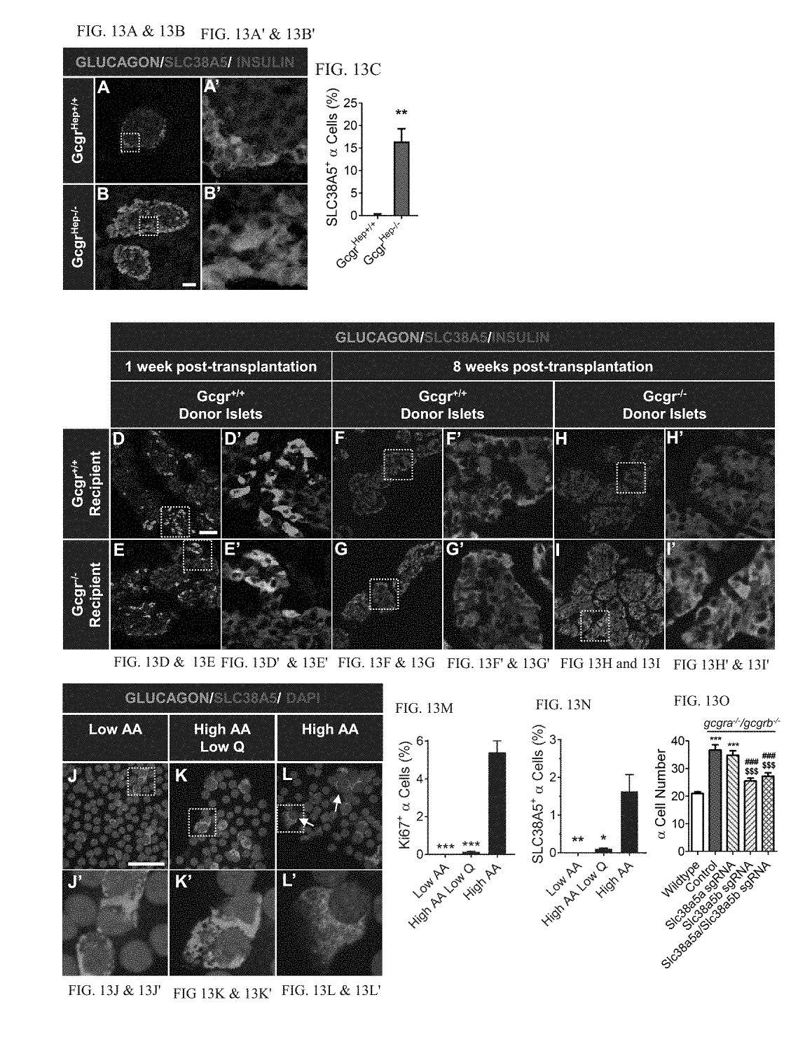

Since mouse endocrine mitogens rarely stimulate proliferation of adult human endocrine cells and mouse and human islets have other substantial, experiments were conducted to determine whether human α-cells proliferate following interruption of glucagon signaling. This is a relevant question since Gcgr inhibitors are in clinical phases of development for the treatment of both type 2 and type 1 diabetes. To investigate whether human α-cell proliferation is increased in response to interruption of glucagon signaling, human islets were transplanted into the subcapsular renal space of NOD.Cg-Prkdc^{scid}Il2rg^{tm1Wjl}Sz (NSG™) immunodeficient mice, which were treated with Gcgr mAb (FIGS. 6A and 12B). As expected, Gcgr mAb-treated mice had lower glycemia, elevated GLP-1, and hyperglucagonemia (FIGS. 6B-D and 12D-G). Gcgr mAb treatment increased α-cell proliferation in both the endogenous mouse pancreatic islets and the transplanted human islets (FIGS. 6E-G and 12I-L). While the majority of human islets showed 5-27 fold increase in α-cell proliferation in response to Gcgr mAb treatment, islets from 2 donors did not (FIG. 6G). Interestingly, the baseline proliferation rates for these two donors (Donor 2 and 3) were 16 fold higher than the baseline proliferation rates in the other 5 human donor islets that responded to Gcgr mAb treatment (Donors 1, 4-7) (FIG. 12N). α-cell proliferation rates in responder and non-responder donor islets were not significantly different after Gcgr mAb [Gcgr mAb-treated α-cell proliferation rate responders (FIG. 12N). Therefore, most human adult α-cells in most human islet preparations proliferate in response to interrupted glucagon signaling.

Elevated Arginine Levels Stimulate Alpha Cell Proliferation

Herein, the contribution of other amino acids to alpha cell proliferation was shown and determined that high levels of arginine stimulated alpha cell proliferation, but not glycine, histidine, asparagine, isoleucine lysine, methionine, valine, alanine, ornithine, proline, serine, or tyrosine.

SLC38A5 Expression is Upregulated in α-Cells and Required for α-Cell Expansion

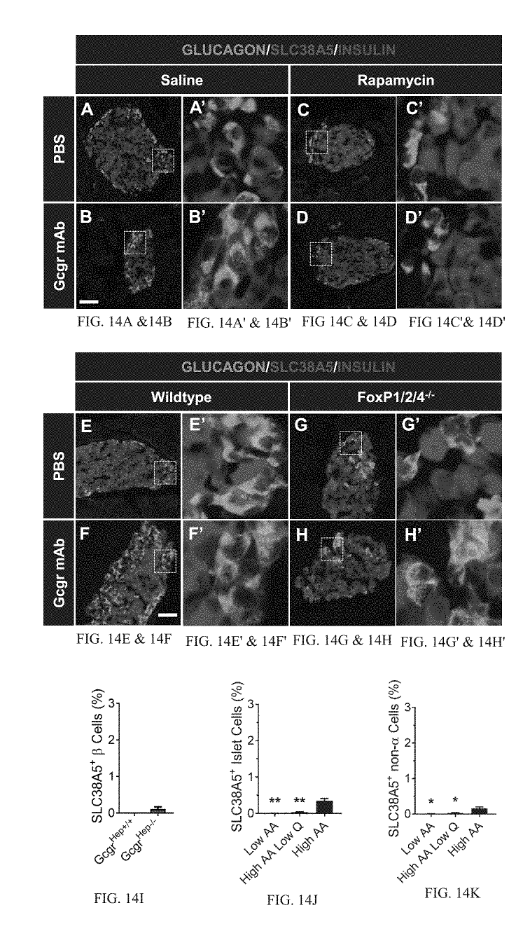

Glutamine transport into α-cells is required for α-cell proliferation and glucagon secretion. The role of the amino acid transporter Slc38a5 in α-cell proliferation was investigated. Mice with interrupted glucagon signaling (Gcgr^{Hep-/-}) had an 82-fold increase in the number of α-cells expressing SLC38A5 over control mice (Gcgr^{HeP+/-}) that rarely had detectable expression in α-cells and weak expression in the exocrine tissue (FIGS. 13A-C). Similarly, α-cells in wildtype mice treated with GCGR mAb had upregulated expression of SLC38A5 and this increase was mitigated by co-treatment with rapamycin (FIG. 14A-D). SLC38A5 is upregulated in α-cells of mice with interrupted glucagon signaling and that this upregulation is sensitive to rapamycin treatment. SLC38A5 expression was occasionally expressed in α-cells of FoxP1/2/4 islet null mice treated with GCGR mAb (FIGS. 14E-H). α-cells of Gcgr+/+ or −/− islets transplanted into Gcgr−/− mice for either one or eight weeks had increased proliferation and robust SLC38A5 expression (FIGS. 13E, 13G, 13I) while those transplanted into Gcgr−/+ mice had undetectable SLC38A5 expression (FIGS. 13D, 13F, 13H). SLC38A5 expression was undetectable in α-cells after 3 days of culture in High amino acid media when α-cell proliferation rates were greater than 4%. However, at 4 days of culture 1.5% of α-cells cultured in high amino acid media (High AA) expressed SLC38A5 (FIGS. 13L-M) while α-cell proliferation rates were 5% (FIG. 13N). Similar to proliferating α-cells, SLC38A5 expression in α-cells was extremely rare in media containing either low amino acids (Low AA) or high amino acids with low levels of glutamine (High AA Low Q) (FIG. 13J-K, 13M-N). Together, these data demonstrate that SLC38A5 expression in α-cells is regulated by amino acids in an mTOR-dependent fashion. To test the role of SLC38A5 in α-cell expansion, CRISPR-mediated knockdown of slc38a5 genes in gcgra^{-/-}/gcgrb^{-/-} zebrafish was used and found that ablation of slc38a5b, but not slc38a5a partially prevented α-cell expansion (FIG. 130). These data support that glutamine uptake into α-cells plays a critical role in α-cells' ability to proliferate in response to high amino acid levels.

Interrupting GCGR signaling by various ways leads to α-cell proliferation, and a hepatic-derived circulating mitogen has been proposed. Using a comprehensive multimodal approach and three models with altered glucagon signaling, it was found that the activity resides in <10 kDa fraction of mouse serum, alterations in genes regulating hepatic amino acid catabolism, and increased serum amino acid levels. Increased amino acids, but not lipids and other soluble factors, selectively increased rapamycin-sensitive α-cell proliferation. Of these elevated amino acids, glutamine and the putative glutamine transporter SLC38A5, play a predominant role. Additionally, FoxP transcription factors in islet α-cells were required to stimulate α-cell proliferation, but were not required for activation of mTOR signaling or SLC38A5 expression. Importantly, human α-cells proliferate in response to interrupted glucagon signaling. Based on these results, it was shown herein that glucagon regulates amino acid catabolism and, via a feedback loop, glutamine regulates glucagon via mTOR/FoxP-dependent control of α-cell proliferation.

Glutamine/Amino Acid Transporters

Slc38a5/SNAT5, Slc38a2/SNAT2, Slc7a7-Slc3a2/LAT1, Slc7a2/CAT2, Slc7a8-Slc3a2/LAT2, and Slc38a9 are selectively expressed in mouse α-cells in the islet, and the latter possibly plays a role in glutamine stimulation of glucagon secretion. Mouse α-cells express SLC38A5 during development, but not during adulthood. Adult mouse α-cells rarely express SLC38A5 protein under normal conditions, but upregulate SLC38A5 under conditions of interrupted glucagon signaling, and that SLC38A5 facilitates expansion of α-cells in a model of interrupted glucagon secretion. Additional glutamine/amino acid transporters (Slc7a14 and Slc38a4/SNAT4) are preferentially expressed in human α-cells when compared to other islet and exocrine cells and also play a role.

Methods

Animals

Global Gcgr−/− mice on C57B16/J background have been described previously. Male 12-20 weeks old NOD. Cg-Prkdc^{scid}Il2rg^{tm1Wjl}Sz (NSG™) immunodeficient mice were received from Jackson Laboratory. All mice were maintained on standard rodent chow under 12-hour light/12-hour dark cycle. All experiments were conducted according to protocols and guidelines approved by the Vanderbilt University Institutional Animal Care and Use Committee. For Gcgr mAb treatments, mice were treated weekly with 10 mg/kg of a humanized monoclonal anti-glucagon receptor antibody (Amgen, Inc) intraperitoneally for 3 days to 6 weeks. For rapamycin treatment of mice, rapamycin was prepared from an oral suspension (1 mg/ml RapaImmune) in saline and mice were injected with either saline or 0.2 mg/kg rapamycin every 3 days for 2 weeks total.

Antibodies and Reagents

Mouse glucagon (Abcam #ab10988), rabbit Ki67 (Abcam #ab15580), rabbit pS6 p235/p236 (Cell Signaling #2211), and rabbit amylase (Sigma) were used for immunohistochemistry. DAPI was purchased from Life Technologies. Recombinant mouse Activin A protein was purchased from R&D Biosystems and biological activity was confirmed by the vendor as the ability to stimulate hemoglobin expression in K562 human chronic myologenous leukemia cells. Recombinant human defensin b1 protein (AbD Serotec) was purchased and biological activity was confirmed by the vendor as the ability to stimulate CD34+ dendritic cell migration. Human hepcidin peptide was synthesized (Peptides International) with cyclization to form 4 disulfide bridges (Cys⁷-Cys²³, Cys¹⁰-Cys¹³, Cys¹¹-Cys¹⁹, Cys¹⁴-Cys²²). Rapamycin (Cayman Chemicals) for islet culture treatments was resuspended in DMSO.

Tissue Preparation and Sectioning for Immunohistochemistry

Pancreata and kidneys containing grafts were retrieved and fixed in 4% paraformaldehyde. The tissue was then embedded in OCT (Tissue Tek) and thin sections were prepared using a cryostat (8 μm thick for pancreas and 5 μm thick for kidney grafts).

Islet Isolation and Culture

Pancreatic islets were isolated from male 8-14 week old C57B16/J mice (Jackson Laboratory, ME) and cultured in various media conditions for 3 days. Unless otherwise stated, all media used for islet culture was Gibco RPMI 1640 with 2.055 mM L-glutamine (Life Technologies) with 10% Fetal Bovine Serum (Atlanta Biologicals), 1% Penicillin/Streptomycin (Gibco), and 5.6 mM D-glucose. For complete amino acid content in each media condition, see Table 1. For serum fractionation, serum was centrifuged for 1 hour at 2000×g at room temperature in a 10 kDa molecular weight cutoff spin column (Millipore, Billerica, Mass.). The flow-through is defined as <10 kDa serum fraction and the remaining volume that did not pass though the column is defined as the >10 kDa serum fraction. For lipid removal, serum was treated with Cleanascite reagent (Biotech Support Group, Monmouth Junction, N.J.) prior to islet culture at a 1:1 ratio according to the vendor's protocol.

Cytospin and Counting

After culture, islets were washed in 2 mM EDTA and dispersed in 0.025% Trypsin-2 mM EDTA (Gibco Life Technologies or HyClone GE Healthcare) for 5-10 minutes by gentle pipetting to obtain single or very small cell clusters. Dispersed islet cells were recovered by centrifugation in RPMI media containing 5.6 mM glucose, 10% FBS, and 1% Penicillin/Streptomycin. The resulting cell pellet was resuspended in 100 ul of media and centrifuged onto a glass slide using a cytospin (Thermo Scientific, Waltham, Mass.) centrifuge. Air-dried slides were stored at −80° C. until use, thawed, and immediately fixed in 4% paraformaldehyde before immunocytochemistry. Slides were mounted with Aqua-Poly/Mount (Warrington, Pa.).

Hepatocyte Cultures

Hepatocytes were isolated from Gcgr−/− mice. Mice were perfused with 50 ml of 0.5 mM EDTA 25 mM HEPES containing HBSS without CaCl₂ or MgCl₂ to flush the liver. The mice were then perfused with 40 ml of 0.03% collagenase P (Roche) in 25 mM HEPES containing HBSS with CaCl₂ and MgCl₂. The liver was removed to 10 ml of collagenase P solution in a petri dish and gently agitated to release hepatocytes from the liver parenchyma. The resulting hepatocyte slurry was filtered through 70 um nylon cell strainer (BD Falcon). After washing to remove debris and other cell types, hepatocytes were plated on Collagen I coated tissue culture plates in 10% FBS containing Hepatocyte Maintenance Media (Lonza CC-3199). After 6 hours, the media was replaced with Hepatocyte Maintenance Media without FBS but containing the Hepatocyte supplement pack (minus Epidermal Growth Factor) (Lonza CC-4182). Conditioned media from 3 days hepatocyte cultures were used to replace 10% of the total islet culture RPMI media volume similarly to Gcgr mouse serum supplementation.

Cell Counting

Cytospin slides were imaged using a Leica Microsystems Epifluorescent Microscope DM1 6000B. Tiled images were analyzed by a cytonuclear algorithm developed using the Imaris Software package (Indica Labs) (FIG. 7A). A glucagon surface was created by mapping glucagon immunoreactivity. DAPI and Ki67 positive cells were identified using a spot mapping algorithm. α-cells were identified by masking the glucagon surface on the DAPI staining and then creating a pseudospot where each α-cell was identified. Colocalization was determined by coregistering glucagon pseudospots with Ki67 spots. Each image was then manually checked for accuracy. For all islet culture experiments, a minimum of 500 α-cells counted per replicate (i.e. n=1) were required to be included in an experiment.

Zebrafish Studies

Wildtype and GcgR1−/−R2−/− zebrafish larvae with Gcg-driven RFP expression were collected on dpf 4 and exposed to 2.5 uM rapamycin for 3 days. Fish larvae were mounted and α-cell number in the primary islet was manually scored via 3D fluorescence microscopy as previously described (Li et 1., 2015).

RNA Sequencing

For RNA isolation, approximately 100 mg punches of liver tissue were stored in RNAlater (Ambion) according to the manufacturer's instructions until isolation using RNAeasy Mini kit with DNase I (Qiagen Hilden, Del.) digestion. RNA purity and quantity were determined by Bioanalyzer. Nripesh working on the rest.

Serum Proteomics and Metabolomics

Whole mouse serum was analyzed for protein expression by SOMAscan® assay (Somalogic, Inc. Boulder, Colo.) aptamer-based detection technique. While over 1400 proteins can be detected in human serum/plasma, ˜400 proteins can be detected in mouse serum/plasma due to aptamer cross-reactivity. For LC-MS/MS analyses of serum, serum was fractionated by centrifugation in a 10 kDa molecular weight cutoff column. The flowthrough was analyzed by SDS-PAGE with Coomassie blue staining. Albumin was removed by albumin antibody-coupled magnetic beads (EMD Millipore) according to vendor instructions. Serum proteins were porcine trypsin digested and identified by LC-MS/MS ionization (Thermo Q-Exactive MS) and analysis using IDPicker 2.0. Serum amino acids were measured by tandem MS.

Human Islet Transplantation

Human islets quality from the Integrated Islet Distribution Program was validated by islet perifusion method. After overnight culture in CRML media, human islets were aliquoted for transplantation. NSG male mice were transplanted with 500 human islets into the subcapsular space of the left kidney. Two weeks after engraftment, mice were injected intraperitoneally with either PBS or Gcgr mAb 10 mg/kg weekly for either 2 or 6 weeks. For total individual donor proliferation rate the total number of α-cells counted in all mice were included for analyses (i.e. a cohort of mice is n=1).

Hormone Assays Measuring Amino Acids

Mice were fasted for 6 hours and blood was collected prior to glucose arginine challenge and blood collection 15 minutes after challenge. Serum was used to measure glucagon by RIA (Millipore) and GLP-1 by Luminex Assay (Millipore) by the Vanderbilt University Hormone Assay Core. For amino acid and acylcarnitine measurements, serum was measured by Tandem MS/MS at the Stedman Center at Duke University using a Quattro Micro instrument (Waters Corporation, Milford, Mass.).

Statistics

Statistical significance was assessed by One-Way or Two-way ANOVA with Tukey multiple comparison post-test or, where appropriate Unpaired Student's T-test using GraphPad Prism 6 (San Diego, Calif.). A p value <0.05 was considered significant.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

TABLE 1
Amino acid media concentrations used for islet cultures model.

| Media # | 1 | 2 | 3 | 4 | 5 | 6 | 7 | 8 | 9 |
|---|---|---|---|---|---|---|---|---|---|
| Glycine | 451 | 200 | 905 | 165 | 210 | 141 | 133 | 270 | 1500 |
| L-Arginine | 158 | 40 | 397 | 1050 | 1074 | 1142 | 1149 | 1094 | 600 |
| L-Asparagine | 27 | 40 | 76 | 344 | 349 | 376 | 379 | 376 | 350 |
| L-Aspartic acid | 50 | 10 | 60 | 140 | 141 | 149 | 150 | 136 | 10 |
| L-Cystine 2HCl | 20 | 10 | 40 | 189 | 191 | 206 | 208 | 188 | 10 |
| L-Glutamic Acid | 50 | 100 | 100 | 127 | 132 | 136 | 136 | 152 | 300 |
| L-Glutamine | 86 | 500 | 283 | 1858 | 1878 | 2037 | 2055 | 2174 | 3250 |
| L-Histidine | 57 | 40 | 170 | 93 | 104 | 98 | 97 | 112 | 250 |
| L-Hydroxyproline | 40 | 0 | 108 | 40 | 108 | 152 | 153 | 137 | 0 |
| L-Isoleucine | 273 | 125 | 317 | 371 | 375 | 381 | 382 | 369 | 250 |
| L-Leucine | 273 | 225 | 317 | 371 | 375 | 381 | 382 | 384 | 400 |
| L-Lysine hydrochloride | 350 | 200 | 1000 | 282 | 347 | 281 | 274 | 367 | 1200 |
| L-Methionine | 106 | 60 | 209 | 101 | 111 | 102 | 101 | 119 | 280 |
| L-Phenylalanine | 69 | 75 | 67 | 89 | 89 | 91 | 91 | 89 | 75 |
| L-Proline | 154 | 85 | 279 | 172 | 184 | 175 | 174 | 197 | 400 |
| L-Serine | 239 | 100 | 959 | 281 | 353 | 292 | 286 | 382 | 1250 |
| L-Threonine | 200 | 150 | 900 | 171 | 241 | 175 | 168 | 326 | 1750 |
| L-Tryptophan | 40 | 65 | 45 | 26 | 27 | 25 | 25 | 29 | 65 |
| L-Tyrosine | 104 | 50 | 196 | 110 | 120 | 112 | 111 | 125 | 250 |
| L-Valine | 265 | 300 | 414 | 180 | 195 | 173 | 171 | 214 | 600 |
| Alanine | 1125 | 350 | 3040 | 113 | 304 | 30 | 0 | 225 | 2250 |
| Ornithine | 86 | 100 | 234 | 9 | 23 | 2 | 0 | 40 | 400 |
| Citrulline | 65 | 0 | 118 | 7 | 12 | 1 | 0 | 0 | 0 |
| Total [Amino acid] | 4288 | 2825 | 10234 | 6289 | 6943 | 6658 | 6625 | 7505 | 15440 |
| concentration in media | | | | | | | | | |

### REFERENCES

Aamodt, K. I., R. Aramandla, J. J. Brown, N. Fiaschi-Taesch, P. Wang, A. F. Stewart, M. Brissova and A. C. Powers (2016). “Development of a reliable automated screening system to identify small molecules and biologics that promote human beta-cell regeneration.” Am J Physiol Endocrinol Metab 311(5): E859-E868.

An, J., D. M. Muoio, M. Shiota, Y. Fujimoto, G. W. Cline, G. I. Shulman, T. R. Koves, R. Stevens, D. Millington and C. B. Newgard (2004). “Hepatic expression of malonyl-CoA decarboxylase reverses muscle, liver and whole-animal insulin resistance.” Nat Med 10(3): 268-274.

Benner, C., T. van der Meulen, E. Caceres, K. Tigyi, C. J. Donaldson and M. O. Huising (2014). “The transcriptional landscape of mouse beta cells compared to human beta cells reveals notable species differences in long non-coding RNA and protein-coding gene expression.” BMC Genomics 15: 620.

Ben-Zvi, D., O. Barrandon, S. Hadley, B. Blum, Q. P. Peterson and D. A. Melton (2015). “Angpt14 links alpha-cell proliferation following glucagon receptor inhibition with adipose tissue triglyceride metabolism.” Proc Natl Acad Sci USA 112: 15498-15503.

Bernal-Mizrachi, E., R. N. Kulkarni, D. K. Scott, F. Mauvais-Jarvis, A. F. Stewart and A. Garcia-Ocana (2014). “Human beta-cell proliferation and intracellular signaling part 2: still driving in the dark without a road map.” Diabetes 63: 819-831.

Bramswig, N. C., L. J. Everett, J. Schug, C. Dorrell, C. Liu, Y. Luo, P. R. Streeter, A. Naji, M. Grompe and K. H. Kaestner (2013). “Epigenomic plasticity enables human pancreatic alpha to beta cell reprogramming.” J Clin Invest 123: 1275-1284.

Brissova, M., K. Aamodt, P. Brahmachary, N. Prasad, J. Y. Hong, C. Dai, M. Mellati, A. Shostak, G. Poffenberger, R. Aramandla, S. E. Levy and A. C. Powers (2014). “Islet microenvironment, modulated by vascular endothelial growth factor-A signaling, promotes beta cell regeneration.” Cell Metab 19: 498-511.

Campbell, J. E. and D. J. Drucker (2015). “Islet alpha cells and glucagon—critical regulators of energy homeostasis.” Nat Rev Endocrinol 11: 329-338.

Cetin, I., S. Ronzoni, A. M. Marconi, G. Perugino, C. Corbetta, F. C. Battaglia and G. Pardi (1996). “Maternal concentrations and fetal-maternal concentration differences of plasma amino acids in normal and intrauterine growth-restricted pregnancies.” Am J Obstet Gynecol 174: 1575-1583.

Chen, T., Y. Ni, X. Ma, Y. Bao, J. Liu, F. Huang, C. Hu, G. Xie, A. Zhao, W. Jia and W. Jia (2016). “Branched-chain and aromatic amino acid profiles and diabetes risk in Chinese populations.” Sci Rep 6: 20594.

Dai, C., M. Brissova, Y. Hang, C. Thompson, G. Poffenberger, A. Shostak, Z. Chen, R. Stein and A. C. Powers (2012). “Islet-enriched gene expression and glucose-induced insulin secretion in human and mouse islets.” Diabetologia 55: 707-718.

Dai, C., N. S. Kayton, A. Shostak, G. Poffenberger, H. A. Cyphert, R. Aramandla, C. Thompson, I. G. Papagiannis, C. Emfinger, M. Shiota, J. M. Stafford, D. L. Greiner, P. L. Herrera, L. D. Shultz, R. Stein and A. C. Powers (2016). “Stress-impaired transcription factor expression and insulin secretion in transplanted human islets.” J Clin Invest 126: 1857-1870.

Dean, E. D., R. H. Unger and W. L. Holland (2017). “Glucagon antagonism in islet cell proliferation.” Proc Natl Acad Sci USA 114: 3006-3008.

DiGruccio M R, et al. (2016) Comprehensive alpha, beta and delta cell transcriptomes reveal that ghrelin selectively activates delta cells and promotes somatostatin release from pancreatic islets. Mol Metab 5(7):449-458.

Dorrell, C., J. Schug, C. F. Lin, P. S. Canaday, A. J. Fox, O. Smirnova, R. Bonnah, P. R. Streeter, C. J. Stoeckert, Jr., K. H. Kaestner and M. Grompe (2011). “Transcriptomes of the major human pancreatic cell types.” Diabetologia 54: 2832-2844.

Economides, D. L., K. H. Nicolaides, W. A. Gahl, I. Bernardini and M. I. Evans (1989). “Plasma amino acids in appropriate- and small-for-gestational-age fetuses.” Am J Obstet Gynecol 161: 1219-1227.

Ferrara, C. T., P. Wang, E. C. Neto, R. D. Stevens, J. R. Bain, B. R. Wenner, O. R. Ilkayeva, M. P. Keller, D. A. Blasiole, C. Kendziorski, B. S. Yandell, C. B. Newgard and A. D. Attie (2008). “Genetic networks of liver metabolism revealed by integration of metabolic and transcriptional profiling.” PLoS Genet 4: e1000034.

Gammelsaeter, R., M. Jenstad, M. K. Bredahl, V. Gundersen and F. A. Chaudhry (2009). “Complementary expression of SN1 and SAT2 in the islets of Langerhans suggests concerted action of glutamine transport in the regulation of insulin secretion.” Biochem Biophys Res Commun 381: 378-382.

Gelling, R. W., X. Q. Du, D. S. Dichmann, J. Romer, H. Huang, L. Cui, S. Obici, B. Tang, J. J. Holst, C. Fledelius, P. B. Johansen, L. Rossetti, L. A. Jelicks, P. Serup, E. Nishimura and M. J. Charron (2003). “Lower blood glucose, hyperglucagonemia, and pancreatic alpha cell hyperplasia in glucagon receptor knockout mice.” Proc Natl Acad Sci USA 100: 1438-1443.

Graham, M. L., J. McCormick, M. Verardo, M. A. Demore, A. A. Anderson, H. Yan, M. J. Charron, R. A. Lindberg, W. Gu, and M. M. Ellison (2008). “Dysregulation of glucagon secretion in glucagon receptor null pancreatic islets.” ADA 68^{th} Scientific Sessions 1660-P.

Gu, W., H. Yan, K. A. Winters, R. Komorowski, S. Vonderfecht, L. Atangan, G. Sivits, D. Hill, J. Yang, V. Bi, Y. Shen, S. Hu, T. Boone, R. A. Lindberg and M. M. Veniant (2009). “Long-term inhibition of the glucagon receptor with a monoclonal antibody in mice causes sustained improvement in glycemic control, with reversible alpha-cell hyperplasia and hyperglucagonemia.” J Pharmacol Exp Ther 331: 871-881.

Guasch-Ferre, M., A. Hruby, E. Toledo, C. B. Clish, M. A. Martinez-Gonzalez, J. Salas-Salvado and F. B. Hu (2016). “Metabolomics in Prediabetes and Diabetes: A Systematic Review and Meta-analysis.” Diabetes Care 39: 833-846.

Hassanein, M., M. D. Hoeksema, M. Shiota, J. Qian, B. K. Harris, H. Chen, J. E. Clark, W. E. Alborn, R. Eisenberg and P. P. Massion (2013). “SLC1A5 mediates glutamine transport required for lung cancer cell growth and survival.” Clin Cancer Res 19: 560-570.

Hensley, C. T., A. T. Wasti and R. J. DeBerardinis (2013). “Glutamine and cancer: cell biology, physiology, and clinical opportunities.” J Clin Invest 123: 3678-3684.

Hoist, J. J., N. J. Wewer Albrechtsen, J. Pedersen and F. K. Knop (2017). “Glucagon and Amino Acids Are Linked in a Mutual Feedback Cycle: The Liver-alpha-Cell Axis.” Diabetes 66: 235-240.

Kayton, N. S., G. Poffenberger, J. Henske, C. Dai, C. Thompson, R. Aramandla, A. Shostak, W. Nicholson, M. Brissova, W. S. Bush and A. C. Powers (2015). “Human islet preparations distributed for research exhibit a variety of insulin-secretory profiles.” Am J Physiol Endocrinol Metab 308: E592-602.

Kim, J., H. Okamoto, Z. Huang, G. Anguiano, S. Chen, Q. Liu, K. Cavino, Y. Xin, E. Na, R. Hamid, J. Lee, B. Zambrowicz, R. Unger, A.J. Murphy, Y. Xu, G.D. Yancopoulos, W. Li, and J. Gromada (2017). “Amino acid transporter Slc38a5 contributes to glucagon receptor inhibtion-induced pancreatic α-cell hyperplasia in mice.”Cell Metab in press.

King, M., T. Pearson, L. D. Shultz, J. Leif, R. Bottino, M. Trucco, M. A. Atkinson, C. Wasserfall, K. C. Herold, R. T. Woodland, M. R. Schmidt, B. A. Woda, M. J. Thompson, A. A. Rossini and D. L. Greiner (2008). “A new Hu-PBL model for the study of human islet alloreactivity based on NOD-scid mice bearing a targeted mutation in the IL-2 receptor gamma chain gene.” Clin Immunol 126(3): 303-314.

Larger, E., N. J. Wewer Albrechtsen, L. H. Hansen, R. W. Gelling, J. Capeau, C. F. Deacon, O. D. Madsen, F. Yakushiji, P. De Meyts, J. J. Holst and E. Nishimura (2016). “Pancreatic alpha-cell hyperplasia and hyperglucagonemia due to a glucagon receptor splice mutation.” Endocrinol Diabetes Metab Case Rep EDM160081.

Li, M., E. D. Dean, L. Zhao, W. E. Nicholson, A. C. Powers and W. Chen (2015). “Glucagon receptor inactivation leads to alpha-cell hyperplasia in zebrafish.” J Endocrinol 227(2): 93-103.

Li, T., Z. Zhang, S. C. Kolwicz, Jr., L. Abell, N. D. Roe, M. Kim, B. Zhou, Y. Cao, J. Ritterhoff, H. Gu, D. Raftery, H. Sun and R. Tian (2017). “Defective Branched-Chain Amino Acid Catabolism Disrupts Glucose Metabolism and Sensitizes the Heart to Ischemia-Reperfusion Injury.” Cell Metab 25: 374-385.

Longuet, C., A. M. Robledo, E. D. Dean, C. Dai, S. Ali, I. McGuinness, V. de Chavez, P. M. Vuguin, M. J. Charron, A. C. Powers and D. J. Drucker (2013). “Liver-specific disruption of the murine glucagon receptor produces alpha-cell hyperplasia: evidence for a circulating alpha-cell growth factor.” Diabetes 62: 1196-1205.

Longuet, C., E. M. Sinclair, A. Maida, L. L. Baggio, M. Maziarz, M. J. Charron and D. J. Drucker (2008). “The glucagon receptor is required for the adaptive metabolic response to fasting.” Cell Metab 8: 359-371.

Miller, H. C., M. Kidd, I. M. Modlin, P. Cohen, R. Dina, P. Drymousis, P. Vlavianos, G. Kloppel and A. Frilling (2015). “Glucagon receptor gene mutations with hyperglucagonemia but without the glucagonoma syndrome.” World J Gastrointest Surg 7: 60-66.

Newgard, C. B. (2012). “Interplay between lipids and branched-chain amino acids in development of insulin resistance.” Cell Metab 15: 606-614.

Ohneda, A., E. Aguilar-Parada, A. M. Eisentraut, and R. H. Unger (1968). “Characterization of response of circulating glucagon to intraduodenal and intravenous administration of amino acids”. J Clin Invest 47: 2305-2322.

Okamoto, H., J. Kim, J. Aglione, J. Lee, K. Cavino, E. Na, A. Rafique, J. H. Kim, J. Harp, D. M. Valenzuela, G. D. Yancopoulos, A. J. Murphy and J. Gromada (2015). “Glucagon Receptor Blockade With a Human Antibody Normalizes Blood Glucose in Diabetic Mice and Monkeys.” Endocrinology 156: 2781-2794.

Okamoto, H., K. Cavino, E. Na, E. Krumm, S. Kim, P. E. Stevis, J. Harp, A. J. Murphy, G. D. Yancopoulos and J. Gromada (2017). “Angptl4 does not control hyperglucagonemia or alpha-cell hyperplasia following glucagon receptor inhibition.” Proc Natl Acad Sci USA 114: 2747-2752.

Parker, J. C., K. M. Andrews, M. R. Allen, J. L. Stock and J. D. McNeish (2002). “Glycemic control in mice with targeted disruption of the glucagon receptor gene.” Biochem Biophys Res Commun 290: 839-843.

Petersson, B. and B. Hellman (1963). “Effects of Long Term Administration of Glucagon on the Pancreatic Islet Tissue of Rats and Guinea-Pigs.” Acta Endocrinol (Copenh) 44: 139-149.

Pochini, L., M. Scalise, M. Galluccio and C. Indiveri (2014). “Membrane transporters for the special amino acid glutamine: structure/function relationships and relevance to human health.” Front Chem 2: 61.

Reusens, B., S. E. Ozanne and C. Remade (2007). “Fetal determinants of type 2 diabetes.” Curr Drug Targets 8: 935-941.

Sayers, S. R., F. Reimann, F. M. Gribble, H. Parker, S. Zac-Varghese, S. R. Bloom, M. Foretz, B. Viollet and G. A. Rutter (2016). “Proglucagon Promoter Cre-Mediated AMPK Deletion in Mice Increases Circulating GLP-1 Levels and Oral Glucose Tolerance.” PLoS One 11: e0149549.

Segerstolpe A, et al. (2016) Single-Cell Transcriptome Profiling of Human Pancreatic Islets in Health and Type 2 Diabetes. Cell Metab 24(4):593-607.

Sheen, J. H., R. Zoncu, D. Kim and D. M. Sabatini (2011). “Defective regulation of autophagy upon leucine deprivation reveals a targetable liability of human melanoma cells in vitro and in vivo.” Cancer Cell 19: 613-628.

Solloway, M. J., A. Madjidi, C. Gu, J. Eastham-Anderson, H. J. Clarke, N. Kljavin, J. Zavala-Solorio, L. Kates, B. Friedman, M. Brauer, J. Wang, O. Fiehn, G. Kolumam, H. Stern, J. B. Lowe, A. S. Peterson and B. B. Allan (2015). “Glucagon Couples Hepatic Amino Acid Catabolism to mTOR-Dependent Regulation of alpha-Cell Mass.” Cell Rep 12: 495-510.

Song, W. J., P. Mondal, A. Wolfe, L. C. Alonso, R. Stamateris, B. W. Ong, O. C. Lim, K. S. Yang, S. Radovick, H. J. Novaira, E. A. Farber, C. R. Farber, S. D. Turner and M. A. Hussain (2014). “Glucagon regulates hepatic kisspeptin to impair insulin secretion.” Cell Metab 19: 667-681.

Spaeth, J. M., C. S. Hunter, L. Bonatakis, M. Guo, C. A. French, I. Slack, M. Hara, S. E. Fisher, J. Ferrer, E. E. Morrisey, B. Z. Stanger and R. Stein (2015). “The FOXP1, FOXP2 and FOXP4 transcription factors are required for islet alpha cell proliferation and function in mice.” Diabetologia 58: 1836-1844.

Takano, Y., K. Kasai, Y. Takagishi, T. Kikumori, T. Imai, Y. Murata and Y. Hayashi (2015). “Pancreatic Neuroendocrine Tumors in Mice Deficient in Proglucagon-Derived Peptides.” PLoS One 10: e0133812.

von Dorsche, H. H. and M. Ziegler (1981). “[A-cell-hyperplasia of islets of Langerhans on rabbits after immunisation with glucagon (author's transl)].” Acta Histochem 68: 290-293.

Watanabe, C., Y. Seino, H. Miyahira, M. Yamamoto, A. Fukami, N. Ozaki, Y. Takagishi, J. Sato, T. Fukuwatari, K. Shibata, Y. Oiso, Y. Murata and Y. Hayashi (2012). “Remodeling of hepatic metabolism and hyperaminoacidemia in mice deficient in proglucagon-derived peptides.” Diabetes 61: 74-84.

Xin, Y., J. Kim, H. Okamoto, M. Ni, Y. Wei, C. Adler, A. J. Murphy, G. D. Yancopoulos, C. Lin and J. Gromada (2016). “RNA Sequencing of Single Human Islet Cells Reveals Type 2 Diabetes Genes.” Cell Metab 24: 608-615.

Yang, J., M. L. MacDougall, M. T. McDowell, L. Xi, R. Wei, W. J. Zavadoski, M. P. Molloy, J. D. Baker, M. Kuhn, O. Cabrera and J. L. Treadway (2011). “Polyomic profiling reveals significant hepatic metabolic alterations in glucagon-receptor (GCGR) knockout mice: implications on anti-glucagon therapies for diabetes.” BMC Genomics 12: 281.

Yin L., L. E. Jao, and W. Chen. (2015) “Generation of targeted mutations in zebrafish using CRISPR/Cas9.” Methods in Cell Biology 1332:205-217.

Yoon, M. S. (2016). “The Emerging Role of Branched-Chain Amino Acids in Insulin Resistance and Metabolism.” Nutrients 8: 405.

Yu, R., Y. Zheng, M. B. Lucas and Y. G. Tong (2015). “Elusive liver factor that causes pancreatic alpha cell hyperplasia: A review of literature.” World J Gastrointest Pathophysiol 6: 131-139.

Zhou, C., D. Dhall, N. N. Nissen, C. R. Chen and R. Yu (2009). “Homozygous P86S mutation of the human glucagon receptor is associated with hyperglucagonemia, alpha cell hyperplasia, and islet cell tumor.” Pancreas 38: 941-946.

Zimmerman, L. J., M. Li, W. G. Yarborough, R. J. C. Slebbos, and D. C. Liebler (2012). “Global Stability of Plasma Proteomes for Mass Spectometry-Based Analyses.” Mol. Cell. Proteomics 11: 1-12.

What is claimed is:

1. A method for treating a subject with diabetes, comprising administering to the subject a composition comprising one or more L-glutamine inhibitors, one or more L-arginine inhibitors, or a combination thereof in an amount effective to decrease blood glucagon levels.

2. The method of claim 1, wherein the L-glutamine inhibitor is a glutaminase (GLS) inhibitor.

3. The method of claim 2, wherein the L-glutamine inhibitor comprises Bis-2-(5-phenylacetamido-1,3,4-thiadiazol-2-ypethyl sulfide (BPTES).

4. The method of claim 1, wherein the L-glutamine inhibitor comprises azaserine or 6-diazo-5-oxo-L-norleucine (L-DON).

5. The method of claim 1, wherein the L-glutamine inhibitor comprises an inhibitor of a SLC 7A5/SLC3A2 transporter.

6. The method of claim 5, wherein the L-glutamine inhibitor comprises 2-aminobicyclo-(2,2,1) heptanecarboxylic acid (BCH).

7. The method of claim 1, wherein the L-glutamine inhibitor comprises an inhibitor of SLC1A5 transporter.

8. The method of claim 7, wherein the L-glutamine inhibitor comprises L-γ-glutamyl-p-nitroanilide (GPNA).

9. The method of claim 1, wherein the L-glutamine inhibitor comprises 4-Phenylbutyrate (4-PBA).

10. The method of claim 1, wherein the L-glutamine inhibitor comprises an Asparaginase.

11. The method of claim 1, wherein the subject has pre-diabetes.

12. The method of claim 1, wherein the subject has type 1 diabetes.

13. The method of claim 1, wherein the subject has type 2 diabetes.

14. The method of claim 1, wherein the subject has gestational diabetes.

15. The method of claim 1, wherein the L-glutamine inhibitor is administered in combination with metaformin or insulin.

16. The method of claim 1, wherein the L-glutamine inhibitor is administered in combination with a GLP-1 agonist, DDP-4 inhibitor, or combination thereof.

17. A method for the screening of a pancreatic-alpha-cell proliferation inducing compound, cotnptising the steps of a) contacting at least one pancreatic alpha-cell with a given compound, and b) testing whether said compound is capable of ki67 or pHH3 gene or protein expression.

18. A method for the screening of a pancreatic-alpha-cell proliferation inducing compound, comprising the steps of a) contacting at least one pancreatic alpha-cell with a given compound, and b) testing whether said compound is capable Edu or BrdU incorporation.

19. A method for expanding alpha cells in culture, comprising contacting the alpha cells with an effective amount of a composition comprising L-glutamine.

20. The method of claim 19, further comprising transdifferentiating the expanded alpha cells into beta cells.

21. The method of claim 20, further comprising transplanting the beta cells into a subject with diabetes.

22. The method of claim 21, wherein the alpha cells are autologous.
